(19) United States
(12) Patent Application Publication
(10) Pub. No.: US 2011/0104161 A1
(43) Pub. Date: May 5, 2011

(54) COMBINATIONS VEGF(R) INHIBITORS AND HEPATOCYTE GROWTH FACTOR (C-MET) INHIBITORS FOR THE TREATMENT OF CANCER

(76) Inventors: Teresa L. Burgess, Ventura, CA (US); Angela Coxon, Moorpark, CA (US); Isabelle Dussault, Westlake Village, CA (US); Paula Kaplan-Lefko, Simi Valley, CA (US); Anthony J. Polverino, Bainbridge Island, WA (US); Darrin Beaupre, Simi Valley, CA (US)

(21) Appl. No.: 12/992,359

(22) PCT Filed: May 14, 2009
(86) PCT No.: PCT/US09/44034

§ 371 Date: Jan. 11, 2011

Related U.S. Application Data
(60) Provisional application No. 61/127,753, filed on May 14, 2008.

(51) Int. Cl. A61K 39/395; A61P 35/00; A61K 31/437; A61K 31/5355

(52) U.S. Cl. 424/133.1; 424/172.1; 514/300; 514/235.5

(57) ABSTRACT

This invention is in the field of pharmaceutical agents and specifically relates to compounds, compositions, uses and methods for treating cancer, by—combining VEGF(R) inhibitors and inhibitors of HGF/SF:c-Met.

### FIELD OF THE INVENTION

This invention is in the field of pharmaceutical agents and specifically relates to compounds, compositions, uses and methods for treating cancer.

### BACKGROUND

Protein kinases represent a large family of proteins which play a central role in the regulation of a wide variety of cellular processes, maintaining control over cellular function. A partial list of such kinases includes ab1, Akt, bcr-ab1, Blk, Brk, Btk, c-kit, c-Met, c-src, c-fms, CDK1, CDK2, CDK3, CDK4, CDK5, CDK6, CDK7, CDK8, CDK9, CDK10, cRaf1, CSF1R, CSK, EGFR, ErbB2, ErbB3, ErbB4, Erk, Fak, fes, FGFR1, FGFR2, FGFR3, FGFR4, FGFR5, Fgr, fit-1, Fps, Frk, Fyn, Hck, IGF-1R, INS-R, Jak, KDR, Lck, Lyn, MEK, p38, PDGFR, PIK, PKC, PYK2, ros, tie, tie2, TRK, Yes, and Zap70. Inhibition of such kinases has become an important therapeutic target.

Certain diseases are known to be associated with deregulated angiogenesis, for example ocular neovascularisation, such as retinopathies (including diabetic retinopathy), age-related macular degeneration, psoriasis, hemangioblastoma, hemangioma, arteriosclerosis, inflammatory disease, such as a rheumatoid or rheumatic inflammatory disease, especially arthritis (including rheumatoid arthritis), or other chronic inflammatory disorders, such as chronic asthma, arterial or post-transplantational atherosclerosis, endometriosis, and neoplastic diseases, for example so-called solid tumors and liquid tumors (such as leukemias).

At the center of the network regulating the growth and differentiation of the vascular system and its components, both during embryonic development and normal growth, and in a wide number of pathological anomalies and diseases, lies the angiogenic factor known as Vascular Endothelial Growth Factor” (VEGF; originally termed ‘Vascular Permeability Factor”, VPF), along with its cellular receptors (see G. Breier et al., Trends in Cell Biology, 6:454-456 (1996)).

VEGF is a dimeric, disulfide-linked 46-kDa glycoprotein related to “Platelet-Derived Growth Factor” (PDGF); it is produced by normal cell lines and tumor cell lines; is an endothelial cell-specific mitogen; shows angiogenic activity in in vivo test systems (e.g. rabbit cornea); is chemotactic for endothelial cells and monocytes; and induces plasminogen activators in endothelial cells, which are involved in the proteolytic degradation of extracellular matrix during the formation of capillaries. A number of isoforms of VEGF are known, which show comparable biological activity, but differ in the type of cells that secrete them and in their heparin-binding capacity. In addition, there are other members of the VEGF family, such as “Placenta Growth Factor”(PlGF) and VEGF-C.

VEGF receptors (VEGFR) are transmembrane receptor tyrosine kinases. They are characterized by an extracellular domain with seven immunoglobulin-like domains and an intracellular tyrosine kinase domain. Various types of VEGF receptor are known, e.g. VEGFR-1 (also known as flt-1), VEGFR-2 (also known as KDR), and VEGFR-3.

A large number of human tumors, especially gliomas and carcinomas, express high levels of VEGF and its receptors. This has led to the hypothesis that the VEGF released by tumor cells stimulates the growth of blood capillaries and the proliferation of tumor endothelium in a paracrine manner and through the improved blood supply, accelerate tumor growth. Increased VEGF expression could explain the occurrence of cerebral edema in patients with glioma. Direct evidence of the role of VEGF as a tumor angiogenesis factor in vivo is shown in studies in which VEGF expression or VEGF activity was inhibited. This was achieved with anti-VEGF antibodies, with dominant-negative VEGFR-2 mutants which inhibited signal transduction, and with antisense-VEGF RNA techniques. All approaches led to a reduction in the growth of glioma cell lines or other tumor cell lines in vivo as a result of inhibited tumor angiogenesis.

Angiogenesis is regarded as an absolute prerequisite for tumors which grow beyond a diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size.

Three principal mechanisms play an important part in the activity of angiogenesis inhibitors against tumors: 1) Inhibition of the growth of vessels, especially capillaries, into avascular resting tumors, with the result that there is no net tumor growth owing to the balance that is achieved between cell death and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells which normally line the vessels. See R. Connell and J. Beebe, Exp. Opin. Ther. Patents, 11:77-114 (2001).

VEGF's are unique in that they are the only angiogenic growth factors known to contribute to vascular hyperpermeability and the formation of edema. Indeed, vascular hyperpermeability and edema that is associated with the expression or administration of many other growth factors appears to be mediated via VEGF production.

Inflammatory cytokines stimulate VEGF production. Hypoxia results in a marked upregulation of VEGF in numerous tissues, hence situations involving infarct, occlusion, ischemia, anemia, or circulatory impairment typically invoke VEGF/VPF-mediated responses. Vascular hyperpermeability, associated edema, altered transendothelial exchange and macromolecular extravasation, which is often accompanied by diapedesis, can result in excessive matrix deposition, aberrant stromal proliferation, fibrosis, etc. Hence, VEGF-mediated hyperpermeability can significantly contribute to disorders with these etiologic features. As such, regulators of angiogenesis have become an important therapeutic target. See Hicklin and Ellis, J. Clin Oncology, 23:1011-1027 (2005).

The hepatocyte growth factor receptor (“c-Met”) is a unique receptor tyrosine kinase shown to be overexpressed in a variety of malignancies. c-Met typically comprises, in its native form, a 190-kDa heterodimeric (a disulfide-linked 50-kDa α-chain and a 145-kDa β-chain) membrane-spanning tyrosine kinase protein (Proc. Natl. Acad. Sci. USA, 84:6379-6383 (1987)). c-Met is mainly expressed in epithelial cells and stimulation of c-Met leads to scattering, angiogenesis, proliferation and metastasis. (See Cytokine and Growth Factor Reviews, 13:41-59 (2002)).

The ligand for c-Met is hepatocyte growth factor (also known as scatter factor, HGF, SF and HGF/SF). HGF is a heterodimeric protein secreted by cells of mesodermal origin (Nature, 327:239-242 (1987); J. Cell Biol., 111:2097-2108 (1990)).

Various biological activities have been described for HGF (Hepatocyte Growth Factor-Scatter Factor (HGF-SF) and the c-Met Receptor, Goldberg and Rosen, eds., Birkhauser Verlag-Basel, 67-79 (1993). The biological effect of HGF/SF may depend in part on the target cell. HGF induces a spectrum of biological activities in epithelial cells, including mitogenesis, stimulation of cell motility and promotion of matrix invasion (Biochem. Biophys. Res. Comm., 122:1450-1459 (1984); Proc. Natl. Acad. Sci. U.S.A., 88:415-419 (1991)). It stimulates the motility and invasiveness of carcinoma cells, the former having been implicated in the migration of cells required for metastasis. HGF can also act as a “scatter factor”, an activity that promotes the dissociation of epithelial and vascular endothelial cells (Nature, 327:239-242 (1987); J. Cell Biol., 111:2097-2108 (1990); EMBO J., 10:2867-2878 (1991); Proc. Natl. Acad. Sci. USA, 90:649-653 (1993)). Therefore, HGF is thought to be important in tumor invasion (Hepatocyte Growth Factor-Scatter Factor (HGF-SF) and the Met Receptor, Goldberg and Rosen, eds., Birkhauser Verlag-Basel, 131-165 (1993)).

HGF and c-Met are expressed at abnormally high levels in a large variety of solid tumors. High levels of HGF and/or c-Met have been observed in liver, breast, pancreas, lung, kidney, bladder, ovary, brain, prostate, gallbladder and myeloma tumors in addition to many others. The role of HGF/c-Met in metastasis has been investigated in mice using cell lines transformed with HGF/c-Met (J. Mol. Med., 74:505-513 (1996)). Overexpression of the c-Met oncogene has also been suggested to play a role in the pathogenesis and progression of thyroid tumors derived from follicular epithelium (Oncogene, 7:2549-2553 (1992)). HGF is a morphogen (Development, 110:1271-1284 (1990); Cell, 66:697-711 (1991)) and a potent angiogenic factor (J. Cell Biol., 119:629-641 (1992)).

Recent work on the relationship between inhibition of angiogenesis and the suppression or reversion of tumor progression shows great promise in the treatment of cancer (Nature, 390:404-407 (1997)), especially the use of multiple angiogenesis inhibitors compared to the effect of a single inhibitor. Angiogenesis can be stimulated by HGF, as well as vascular endothelial growth factor (VEGF) and basic fibroblast growth factor (bFGF).

Angiogenesis, the process of sprouting new blood vessels from existing vasculature and arteriogenesis, the remodeling of small vessels into larger conduit vessels are both physiologically important aspects of vascular growth in adult tissues. These processes of vascular growth are required for beneficial processes such as tissue repair, wound healing, recovery from tissue ischemia and menstrual cycling. They are also required for the development of pathological conditions such as the growth of neoplasias, diabetic retinopathy, rheumatoid arthritis, psoriasis, certain forms of macular degeneration, and certain inflammatory pathologies. The inhibition of vascular growth in these contexts has also shown beneficial effects in preclinical animal models. For example, inhibition of angiogenesis by blocking vascular endothelial growth factor or its receptor has resulted in inhibition of tumor growth and in retinopathy. Also, the development of pathological pannus tissue in rheumatoid arthritis involves angiogenesis and might be blocked by inhibitors of angiogenesis.

The ability to stimulate vascular growth has potential utility for treatment of ischemia-induced pathologies such as myocardial infarction, coronary artery disease, peripheral vascular disease, and stroke. The sprouting of new vessels and/or the expansion of small vessels in ischemic tissues prevents ischemic tissue death and induces tissue repair. Certain diseases are known to be associated with deregulated angiogenesis, for example ocular neovascularization, such as retinopathies (including diabetic retinopathy), age-related macular degeneration, psoriasis, hemangioblastoma, hemangioma, arteriosclerosis, inflammatory disease, such as a rheumatoid or rheumatic inflammatory disease, especially arthritis (including rheumatoid arthritis), or other chronic inflammatory disorders, such as chronic asthma, arterial or post-transplantational atherosclerosis, endometriosis, and neoplastic diseases, for example so-called solid tumors and liquid tumors (such as leukemias). Treatment of malaria and related viral diseases may also be mediated by HGF and c-Met.

Elevated levels of HGF and c-Met have also been observed in non-oncological settings, such as hypertension, myocardial infarction and rheumatoid arthritis. It has been observed that levels of HGF increase in the plasma of patients with hepatic failure (Gohda et al., supra) and in the plasma (Hepatol., 13:734-750 (1991)) or serum (J. Biochem., 109:8-13 (1991)) of animals with experimentally induced liver damage. HGF has also been shown to be a mitogen for certain cell types, including melanocytes, renal tubular cells, keratinocytes, certain endothelial cells and cells of epithelial origin (Biochem. Biophys. Res. Commun., 176:45-51 (1991); Biochem. Biophys. Res. Commun, 174:831-838 (1991); Biochem., 30:9768-9780 (1991); Proc. Natl. Acad. Sci. USA, 88:415-419 (1991)). Both HGF and the c-Met proto-oncogene have been postulated to play a role in microglial reactions to CNS injuries (Oncogene, 8:219-222 (1993)).

Metastatic SCC cells overexpress c-Met and have enhanced tumoregenesis and metastasis in vivo [G. Gong et al., Oncogene, 23:6199-6208 (2004)]. C-Met is required for tumor cell survival [N. Shinomiya et al., Cancer Research, 64:7962-7970 (2004)]. For a general review see C. Birchmeier et al., Nature Reviews/Molecular Biology 4:915-925 (2003).

In view of the role of HGF/SF and/or c-Met in potentiating or promoting such diseases or pathological conditions, it would be useful to have a means of substantially reducing or inhibiting one or more of the biological effects of HGF and its receptor.

It is now found that some combinations of a VEGF pathway inhibitor and HGF/SF:c-Met pathway inhibitor provides better results than one or the other inhibitor used alone.

### DESCRIPTION OF THE DRAWINGS

FIG. 1 shows the combination of VEGFR inhibitor, motesanib, and HGF/SF:c-Met inhibitor AMG 102, in the treatment of U118KR human glioblastoma cells.

FIG. 2 shows the combination of VEGFR inhibitor, motesanib, and HGF/SF:c-Met inhibitor AMG 102, in the treatment of U-87 MG human glioblastoma tumor cells.

FIGS. 3A and 3B show the combination of VEGFR inhibitor, Amgen Compound 1, and HGF/SF:c-Met inhibitor, Compound X, in the treatment of MKN45 human gastric carcinoma cells.

FIG. 4 shows a graph of the post-dose tumor response in patients receiving various doses of AMG 102 in combination with motesanib or bevacizumab who had a baseline tumor assessment and at least one post-dose tumor assessment (quantified at study sites as the longest diameters for up to ten target lesions).

FIG. 5 shows the combination of VEGFR inhibitor, motesanib, and HGF/SF:c-Met inhibitor, Amgen Compound 3, in the treatment of MKN45 human gastric carcinoma cells.

FIGS. 6 and 7 show the combination of VEGFR inhibitor, motesanib, and HGF/SF:c-Met inhibitor, Amgen Compound 3, in the treatment of 786-0 human renal cell adenocarcinoma cells.

### DETAILED DESCRIPTION OF THE INVENTION

The present invention is generally directed to compositions and methods for reducing tumor growth, and generally treating tumors in humans. The approach taken by the inventors was to determine whether a combination of HGF/SF:c-Met inhibiting agents with VEGFR inhibiting agents that target the tumor vasculature provides a beneficial effect. The results obtained by the inventors indicate a surprising benefit from the combination of HGF/SF:c-Met inhibiting agents and VEGFR inhibiting agents, and those therapies which involve administration of combinations of these agents are beneficial in the treatment of cancer. Taken individually, the surprising benefits between the individual agents tested provide a number of unforeseen options for the treatment of tumors or cancers.

The invention also relates to treatment of neoplasia including cancer and metastasis, including, but not limited to: carcinoma such as cancer of the bladder, breast, colon (including colorectal cancer), kidney (including renal cell carcinoma), head and neck cancer, including Glioblastoma Multiformae (GBM), liver, lung (including non-small cell lung cancer), esophagus, gall-bladder, ovary, pancreas, stomach, cervix, thyroid, prostate, and skin (including squamous cell carcinoma); hematopoietic tumors of lymphoid lineage (including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g. soft tissue and bone); tumors of the central and peripheral nervous system (including astrocytoma, neuroblastoma, glioma and schwannomas); and other tumors (including melanoma, seminoma, teratocarcinoma, osteosarcoma, xenoderoma pigmentosum, keratoctanthoma, thyroid follicular cancer and Kaposi's sarcoma).

The invention also relates to the treatment of neoplasia selected from lung cancer, including non-small lung cancer, breast cancer, colon cancer, including colorectal cancer, kidney cancer, including renal cell carcinoma, and head and neck cancer, including Glioblastoma Multiforme (GBM).

The invention also relates to the use of the combination of HGF/SF:c-Met inhibiting agents with VEGFR inhibiting agents in adjuvant or neoadjuvant chemotherapy, with or without radiation, for the treatment of neoplasia. “Adjuvant chemotherapy” is defined as the continued treatment after either intensive cycles of chemotherapy and/or radiation, or alternatively after surgery to remove tumors. Alternatively the term describes the use of drugs as additional treatment for patients with cancers that are thought to have spread outside their original sites. Neo-adjuvant therapy is defined as intensive cycles of chemotherapy and/or radiation given to reduce the size of tumor before a definitive surgery. Such adjuvant or neo-adjuvant chemotherapy+/−radiation relates to the treatment of neoplasea including, but not limited to: carcinoma of the breast, colon, kidney, lung, and head and neck.

The invention relates to combinations with VEGFR inhibitors including
N-(4-chlorophenyl)-4-(4-pyridinylmethyl)-1-phthalazinamine;
N-(4-(1,1-dimethylethyl)phenyl)-2-((4-pyridinylmethyl)amino)-3-pyridinecarboxamide;
4-[4-[[[[4-chloro-3-(trifluoromethyl)phenyl]amino]carbonyl]amino]phenoxy]-N-methyl-2-pyridinecarboxamide;
N-[2-(diethylamino)ethyl]-5-[(5-fluoro-1,2-dihydro-2-oxo-3H-indol-3-ylidene)methyl]-2,4-dimethyl-1H-pyrrole-3-carboxamide;
3-[(4-bromo-2,6-difluorophenyl)methoxy]-5-[[[[4-(1-pyrrolidinyl)butyl]amino]carbonyl]amino]-4-isothiazolecarboxamide;
N-(4-bromo-2-fluorophenyl)-6-methoxy-7-[(1-methyl-4-piperidinyl)methoxy]-4-quinazolinamine;
3-[5,6,7,13-tetrahydro-9-[(1-methylethoxy)methyl]-5-oxo-12H-indeno[2,1-a]pyrrolo[3,4-c]carbazol-12-yl]propyl ester N,N-dimethyl-glycine;
N-[5-[[[5-(1,1-dimethylethyl)-2-oxazolyl]methyl]thio]-2-thiazolyl]-4-piperidinecarboxamide;
N-[3-chloro-4-[(3-fluorophenyl)methoxy]phenyl]-6-[5-[[[2-(methylsulfonyl)ethyl]amino]methyl]-2-furanyl]-4-quinazolinamine
4-[(4-methyl-1-piperazinyl)methyl]-N-[4-methyl-3-[[4-(3-pyridinyl)-2-pyrimidinyl]amino]-phenyl]benzamide
N-(3-chloro-4-fluorophenyl)-7-methoxy-6-[3-(4-morpholinyl)propoxy]-4-quinazolinamine
N-(3-ethynylphenyl)-6,7-bis(2-methoxyethoxy)-4-quinazolinamine
N-(3-((((2R)-1-methyl-2-pyrrolidinyl)methyl)oxy)-5-(trifluoromethyl)phenyl)-2-((3-(1,3-oxazol-5-yl)phenyl)amino)-3-pyridinecarboxamide;
2-(((4-fluorophenyl)methyl)amino)-N-(3-((((2R)-1-methyl-2-pyrrolidinyl)methyl)oxy)-5-(trifluoromethyl)phenyl)-3-pyridinecarboxamide;
N-[3-(azetidin-3-ylmethoxy)-5-trifluoromethyl-phenyl]-2-(4-fluoro-benzylamino)-nicotinamide.
6-fluoro-N-(4-(1-methylethyl)phenyl)-2-((4-pyridinylmethyl)amino)-3-pyridinecarboxamide;
2-((4-pyridinylmethyl)amino)-N-(3-(((2S)-2-pyrrolidinylmethyl)oxy)-5-(trifluoromethyl)phenyl)-3-pyridinecarboxamide;
N-(3-(1,1-dimethylethyl)-1H-pyrazol-5-yl)-2-((4-pyridinylmethyl)amino)-3-pyridinecarboxamide;
N-(3,3-dimethyl-2,3-dihydro-1-benzofuran-6-yl)-2-((4-pyridinylmethyl)amino)-3-pyridinecarboxamide;
N-(3-((((2S)-1-methyl-2-pyrrolidinyl)methyl)oxy)-5-(trifluoromethyl)phenyl)-2-((4-pyridinylmethyl)amino)-3-pyridinecarboxamide;
2-((4-pyridinylmethyl)amino)-N-(3-((2-(1-pyrrolidinyl)ethyl)oxy)-4-(trifluoromethyl)phenyl)-3-pyridinecarboxamide;
N-(3,3-dimethyl-2,3-dihydro-1H-indol-6-yl)-2-((4-pyridinylmethyl)amino)-3-pyridinecarboxamide;
N-(4-(pentafluoroethyl)-3-(((2S)-2-pyrrolidinylmethyl)oxy)phenyl)-2-((4-pyridinylmethyl)amino)-3-pyridinecarboxamide;
N-(3-((3-azetidinylmethyl)oxy)-5-(trifluoromethyl)phenyl)-2-((4-pyridinylmethyl)amino)-3-pyridinecarboxamide;
N-(3-(4-piperidinyloxy)-5-(trifluoromethyl)phenyl)-2-((2-(3-pyridinyl)ethyl)amino)-3-pyridinecarboxamide;
N-(4,4-dimethyl-1,2,3,4-tetrahydro-isoquinolin-7-yl)-2-(1H-indazol-6-ylamino)-nicotinamide;
2-(1H-indazol-6-ylamino)-N-[3-(1-methylpyrrolidin-2-ylmethoxy)-5-trifluoromethyl-phenyl]-nicotinamide;
N-[1-(2-dimethylamino-acetyl)-3,3-dimethyl-2,3-dihydro-1H-indol-6-yl]-2-(1H-indazol-6-ylamino)-nicotinamide;
2-(1H-indazol-6-ylamino)-N-[3-(pyrrolidin-2-ylmethoxy)-5-trifluoromethyl-phenyl]-nicotinamide;
N-(1-acetyl-3,3-dimethyl-2,3-dihydro-1H-indol-6-yl)-2-(1H-indazol-6-ylamino)-nicotinamide;
N-(4,4-dimethyl-1-oxo-1,2,3,4-tetrahydro-isoquinolin-7-yl)-2-(1H-indazol-6-ylamino)-nicotinamide;
N-[4-(tert-butyl)-3-(3-piperidylpropyl)phenyl][2-(1H-indazol-6-ylamino)(3-pyridyl)]carboxamide;
N-[5-(tert-butyl)isoxazol-3-yl][2-(1H-indazol-6-ylamino)(3-pyridyl)]carboxamide;
5-fluoro-N-(2-methyl-1,3-benzothiazol-5-yl)-2-((1H-pyrrolo[2,3-b]pyridin-4-ylmethyl)amino)-3-pyridinecarboxamide;
2-(7-isoquinolinylamino)-N-(3-methyl-4-(1-methylethyl)phenyl)-3-pyridinecarboxamide; and
N-[4-(tert-butyl)phenyl][2-(1H-indazol-6-ylamino)(3-pyridyl)]carboxamide.

The invention also relates to a combination with the VEGFR inhibitor motesanib (AMG706).

The invention also relates to combinations with VEGFR inhibitors including AVASTIN® (bevacizumab), NEXAVAR® (sorafenib) (Bayer BAY 43-9006), RECENTIN™ (cediranib) (Astra Zeneca AZ 2171), Novartis/Schering PTK/ZK (vatalanib), PTK787/ZK 222584, Pfizer AG-13736 (axitinib) and SUTENT® (sunitinib) (Pfizer SU11248).

Other VEGFR inhibitors described in the following patents and patent applications can be used in combination therapies: U.S. Pat. No. 6,563,618, US 2003/0166011, US 2006/0223133, PCT/JP1998/05697, US 2006/0241115, WO 2005/070891, U.S. Pat. No. 6,258,812, US 2003/0105091, WO 01/37820, U.S. Pat. No. 6,235,764, WO 01/32651, U.S. Pat. No. 6,630,500, U.S. Pat. No. 6,515,004, U.S. Pat. No. 6,713,485, U.S. Pat. No. 5,521,184, U.S. Pat. No. 5,770,599, U.S. Pat. No. 5,747,498, WO 02/68406, WO 02/66470, WO 02/55501, WO 04/05279, WO 04/07481, WO 04/07458, WO06/012374, WO06/116713, WO 04/09784, WO 02/59110, WO 99/45009, WO 00/59509, WO 99/61422, U.S. Pat. No. 5,990,141, WO 00/12089 and WO 00/02871 each of which is herein incorporated by reference in its entirety, particularly in parts disclosing VEGF inhibitors.

The invention also relates to combinations with VEGFR inhibitors described in US 2003/0125339 or US 2003/0225106 each of which is herein incorporated by reference in its entirety, particularly in parts disclosing VEGF inhibitors.

The invention also relates to combinations with VEGFR inhibitors described in WO 00/42012, WO 00/41698, US 2005/0038080A1, US 2003/0125359A1, US 2002/0165394A1, US 2001/003447A1, US 2001/0016659A1, and US 2002/013774A1 which are herein incorporated by reference in their entirety, particularly in parts disclosing the foregoing VEGF inhibitors.

The invention also relates to combinations with HGF/SF:cMet inhibitors of the formula I

R—X—W—Y—R¹ I

enantiomers, diastereomers, salts solvates, and N-oxides thereof wherein
R is

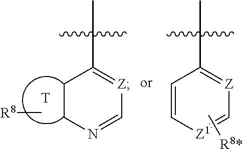

T is selected from phenyl, 5-6-membered heteroaryl, or 5-6 membered heterocyclyl;
Z is selected from N or CR²;
Z¹ is selected from N or CR⁷;
W is an substituted or unsubstituted phenyl, a substituted or unsubstituted benzomorpholinyl, a substituted or unsubstituted 6-membered nitrogen containing heteroaryl; a substituted or unsubstituted c₃₋₇cycloalkyl, c₁₋₆alkyl and c₁₋₆alkynyl;
X is selected from O, S, S(═O), SO₂, NR² and CR³R⁴;
Y is selected from —NR^{a}C(═O)—(CR³R⁴)ₚ—, —NR^{a}C(═S)—(CR³R⁴)ₚ—, —NR^{a}—(CR³R⁴)ₚ—, —NR^{a}—(CR³R⁴)ₚC(═O)—, —NR^{a}—(CR³R⁴)ₚC(═S)—, —NR^{a}S(═O)ₜ—, —NR^{a}S(═O)ₜ—(CR³R⁴)ₚ—, —C(═O)NR^{a}—(CR³R⁴)ₚ—, and —NR^{a}—(CR³R⁴)ₚ—S(═O)ₜ—, and where W is benzomorpholinyl Y may further include —C(═O);
R^{a} is selected from H, alkyl, heterocyclyl, aryl, arylalkyl, heterocyclylalkyl, cycloalkyl, cycloalkylalkyl, alkenyl and alkynyl; wherein R^{a} is optionally substituted;
R¹ is a partially unsaturated or saturated ring selected from

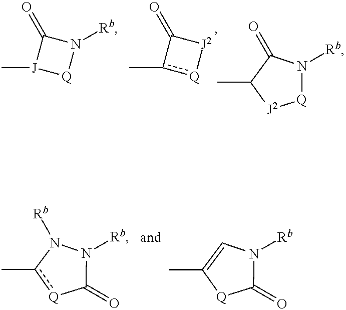

wherein J is N or CR^{4a};
J² is O or CR^{4a}R^{4a};
   Q is a 1-5 membered saturated or partially unsaturated alkyl chain, or a 2-5 membered saturated or partially unsaturated heteroalkyl chain;
R¹ is optionally fused with an optionally substituted phenyl or an optionally substituted 5-6 membered heterocyclyl ring;
wherein R¹ is optionally substituted with one or more substituents independently selected from H, halo, hydroxyl, R^{5a}R^{a}N—, R^{5a}R^{a}N—C₁₋₆ alkyl, R⁵(S═O)—C₁₋₆ alkyl, NR⁵R^{5a}—(C═O)—C₁₋₆ alkyl, optionally substituted alkyl, alkenyl hydroxyalkyl, C₁₋₆ alkoxy-C₁₋₆ alkyl, alkenylalkyl, C₁₋₆ alkylthio-C₁₋₃ alkyl, —C₁₋₆ alkyl-NR^{a}—C(═O)—OR⁵, —C₁₋₃ alkyl-NR^{a}—(C═O)—R⁵, —C₁₋₃ alkyl-C(═O)—C₁₋₃ alkyl, aminoalkyl, hydroxy-substituted aminoalkyl, hydroxy-substituted haloalkyl, (heterocyclo)hydroxyalkyl, haloC₁₋₆-alkyl, azidoalkyl, optionally substituted aryl-C₁₋₆ alkyl, optionally substituted 5-6-membered heterocyclyl-C₁₋₆ alkyl, optionally substituted C₁₋₆-alkyl, optionally substituted C₃₋₇ cycloalkyl, optionally substituted 5-6 membered heterocyclyl, optionally substituted 5-10 membered heteroaryl, optionally, optionally substituted C₃₋₆ cycloalkyl, substituted heteroarylalkyl, optionally substituted arylalkyl, and optionally substituted C₆₋₁₀ aryl;
R^{b} is independently selected at each occurrence from H, optionally substituted arylalkyl, optionally substituted 5-6-membered heterocyclyl-C₁₋₃ alkyl, optionally substituted C₁₋₆-alkyl, optionally substituted 5-6 membered heterocyclyl, optionally substituted C₆₋₁₀ aryl, optionally substituted C₆-10 heteroaryl, optionally substituted C₃₋₆ cycloalkyl, and R^{a}R^{5a}N—C₁₋₃alkyl;

R² is selected from H, alkyl, haloalkyl, aryl, heterocyclyl, arylalkyl, heterocyclylalkyl, cycloalkyl, cycloalkylalkyl, alkenyl, alkynyl and R⁵-carbonyl;
R³ and R⁴ are each independently selected from H, alkyl, aryl, heterocyclyl, arylalkyl, heterocyclylalkyl, haloalkyl, cycloalkyl, cycloalkylalkyl, R⁶ and alkyl substituted with R⁶; alternatively R³ and R⁴, together with the carbon atom they are attached to, form an optionally substituted 3-6 membered ring;
R^{4a} is absent or is selected from H, halo, —OR⁵—NR^{a}R⁵, alkyl, aryl, heterocyclyl, arylalkyl, heterocyclylalkyl, cycloalkyl, cycloalkylalkyl, R⁶ and alkyl substituted with R⁶;
R⁵ is independently selected at each occurrence from H, alkyl, haloalkyl, hydroxyalkyl, alkoxyalkyl, alkylaminoalkyl, alkylthioalkyl, arylalkyl, heterocyclylalkyl, cycloalkylalkyl, aryl, heterocyclyl, alkenyl, alkynyl and cycloalkyl;
R^{5a} is independently selected at each occurrence from H, alkyl, haloalkyl, arylalkyl aminoalkyl, heterocyclylalkyl, cycloalkylalkyl, aryl, heterocyclyl, alkenyl, alkynyl and cycloalkyl;
or when R⁵ and R^{a}, or R^{5a} and R^{a} are bonded to the same nitrogen atom, R^{a} and R⁵, or R^{a} and R^{5a} may independently optionally combine to form a heterocyclo ring.
R⁶ is selected from cyano, —OR², —SR², halo, —SO₂R², —C(═O)R², —SO₂NR²R⁵, —NR⁵C(═O)OR², —NR⁵C(═O)NR⁵R², —NR⁵C(═O)R², —CO₂R², —C(═O)NR²R⁵ and —NR²R⁵;
   R⁷ is selected from H, halo, cyano, —C(═O)NR^{a}R⁵ and alkyl;
   R⁸ is one or more substituents independently selected at each occurrence from H, cyano, hydroxyl, halo, optionally substituted heterocyclyl, —C(═O)NR^{a}R⁵, —OC(═O)NR^{a}R⁵, —NR^{a}C(═O)OR⁵, —NR^{a}C(═O)—R⁵, R⁵R^{a}N—O₂S—, R⁵O₂S—, R⁵O₂SR^{a}N—, R⁵R^{a}N—, alkyl, aminoalkyl, alkylaminoalkyl, alkoxyalkyl, phenylalkyl, heterocyclylalkyl, alkoxy, haloalkoxy, alkylaminoalkoxy, arylalkoxy, heterocyclylalkoxy, cycloalkylalkoxy, heterocyclyl(hydroxyalkoxy), cycloalkyl(hydroxyalkoxy), aryl(hydroxyalkoxy), alkoxyalkoxy, aryloxyalkoxy, heterocyclyloxyalkoxy, cycloalkyloxyalkoxy, aryloxy, heterocyclyloxy, cycloalkyloxy; aryl and heteroaryl, alternatively where R⁸ comprises an NR^{a}R⁵ moiety R^{a} and R⁵, together with the nitrogen atom they are attached to, may optionally form a substituted or unsubstituted 4-6 membered ring;
   R⁸* is one or more substituents independently selected at each occurrence from H, cyano, hydroxyl, halo, optionally substituted heterocyclyl, —NR^{a}C(═O)NR^{a}R⁵′NR^{a}C(═NR^{b})—NR^{a}R⁵, NR^{a}C(═S)NR^{a}R⁵, —OC(═O)NR^{a}R⁵, —NR^{a}C(═O)OR⁵, —NR^{a}C(═O)—R⁵, R⁵R^{a}N—O₂S—, R⁵O₂S—, R⁵O₂SR^{a}N—, R⁵R^{a}N—, alkyl, aminoalkyl, alkylaminoalkyl, alkoxyalkyl, phenylalkyl, heterocyclylalkyl, alkoxy, haloalkoxy, alkylaminoalkoxy, arylalkoxy, heterocyclylalkoxy, cycloalkylalkoxy, heterocyclyl(hydroxyalkoxy), cycloalkyl(hydroxyalkoxy), aryl(hydroxyalkoxy), alkoxyalkoxy, aryloxyalkoxy, heterocyclyloxyalkoxy, cycloalkyloxyalkoxy, aryloxy, heterocyclyloxy, and cycloalkyloxy; alternatively where R⁸* comprises an NR^{a}R⁵ moiety R^{a} and R⁵, together with the nitrogen atom they are attached to, may optionally form a substituted or unsubstituted 4-6 membered ring;
p is 0, 1, 2, or 3; and
t is 0, 1 or 2;
wherein each alkyl, aryl, heteroaryl, cycloalkyl, alkenyl, alkynyl, heterocyclyl, and alkoxy moiety of any R, R¹, R², R³, R⁴, R⁵, R⁷, R⁸, R⁸*, and R^{a} is optionally independently substituted with one or more groups independently selected at each occurrence from halo, oxo, —NR^{a}R⁵, —OR^{5a}, —CO₂R⁵, —C(═O)R⁵, (C₁-C₆)alkylamino, —NH—N═NH, (C₁-C₆)alkyl, (C₁-C₆)alkynyl, (C₃-C₆)cycloalkyl, (C₁-C₆)haloalkyl, di(C₁-C₆)alkylamino, (C₁-C₆)alkylamino-(C₁-C₆)alkyl, (C₁-C₆)hydroxyalkylamino, (C₁-C₆)alkylamino-(C₁-C₆)alkylamino, phenyl, heterocyclic, heteroaryl, —(CR³R⁴)ₚalkyl-S(═O)-alkyl, and —(CR³R⁴)ₚalkyl-S(O)₂-alkyl.

The invention also relates to combinations with HGF/SF:c-Met inhibitors of the formula II

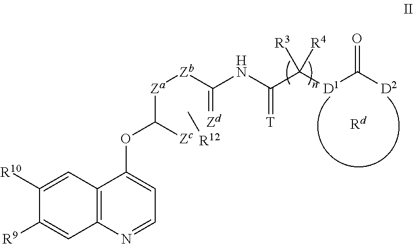

enantiomers, diastereomers, salts, solvates and N-Oxides thereof
wherein T is O or S;
wherein R³ and R⁴ is each independently selected from H, C₁₋₂-alkyl, phenyl, 5-6-membered heterocyclyl, phenyl-C₁₋₂-alkyl, 5-6-membered heterocyclyl-C₁₋₂-alkyl, C₃₋₆-cycloalkyl, and C₃₋₆-cycloalkyl-C₁₋₂-alkyl; alternatively R³ and R⁴, together with the atom they are attached to, form an optionally substituted 3-6 membered ring;
wherein R⁹ and R¹⁰ is independently selected from H, cyano, hydroxy, —C(═O)NR^{a}R^{5a}, 5-6 membered heterocyclyl, —NR^{a}C(═O)—R^{5a}, R^{5a}R^{a}N—O₂S—, R^{5a}O₂SR^{a}N—, R^{5a}R^{a}N—, C₁₋₆-alkyl, amino-C₁₋₆-alkyl, C₁₋₆-alkylamino-C₁₋₆-alkyl, alkoxy-C₁₋₆-alkyl, hydroxy, aryl-C₁₋₆-alkyl, heterocyclyl-C₁₋₆-alkyl, C₁₋₆-alkoxy, halo-C₁₋₆-alkoxy, C₁₋₆-alkylamino-C₁₋₆-alkoxy, aryl-C₁₋₆-alkoxy, 5-6-membered heterocyclyl, —C₁₋₆alkoxy, C₃₋₆-cycloalkyl-C₁₋₆-alkoxy, 5-6-membered heterocyclyl(hydroxyl-C₁₋₆-alkoxy), C₃₋₆-cycloalkyl(hydroxyl-C₁₋₆-alkoxy), phenyl(hydroxyl-C₁₋₆-alkoxy), C₁₋₆-alkoxy-C₁₋₆-alkoxy, phenyloxy-C₁₋₆-alkoxy, 5-6 membered heterocyclyloxy-C₁₋₆-alkoxy, C₃₋₆-cycloalkyloxy-C₁₋₆-alkoxy, phenyloxy, 5-6-membered heterocyclyloxy, and C₃₋₆-cycloalkyloxy;
wherein each of Z^{a}, Z^{b}, Z^{c} and Z^{d} is independently selected from N or CH; provided no more than 2 of Z^{a}, Z^{b}, Z^{c} and Z^{d} are N;
wherein n is 0, 1, 2 or 3;
wherein D¹ is selected from N or CR¹¹;
wherein D² is selected from NR¹³, O, or CHR¹¹; provided either D¹ is N or D² is NR¹³;
wherein ring R^{d} including

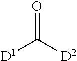

forms an optionally substituted optionally benzo-fused 4-7 membered heterocyclic moiety,
wherein R¹¹ is selected from H, halo, C₁₋₄-alkyl, C₁₋₄-haloalkyl, C₁₋₄-hydroxyalkyl, —NH₂, —OR¹², alkoxycarbonyl, —CO₂H, —CONR³R^{5a}, (C₁-C₃)alkylamino, di(C₁-C₆)alkylamino, (C₁-C₃)hydroxyalkylamino, (C₁-C₃)alkylamino-(C₁-C₃)alkylamino, C₁₋₃-alkoxy-C₁₋₃-alkyl, C₁₋₃-alkylamino-C₁₋₃-alkyl, C₁₋₃-alkylthio-C₁₋₃-alkyl, optionally substituted phenyl-C₁₋₃-alkyl, 5-6 membered heterocyclyl-C₁₋₃-alkyl, C₃₋₆-cycloalkyl-C₁₋₃-alkyl, optionally substituted phenyl, optionally substituted 5-6 membered heterocyclyl, and C₃₋₆-cycloalkyl;
wherein R^{a} is selected from H, alkyl, heterocyclyl, aryl, arylalkyl, heterocyclylalkyl, cycloalkyl, cycloalkylalkyl, alkenyl and alkynyl;
wherein R^{5a} is selected from H, alkyl, haloalkyl, arylalkyl, heterocyclylalkyl, cycloalkylalkyl, aryl, heterocyclyl, alkenyl, alkynyl and cycloalkyl;
wherein R¹² is selected from H, halo, C₁₋₂-alkyl and methoxy;
wherein R¹³ is selected from H, alkyl, haloalkyl, optionally substituted phenylalkyl, optionally substituted 5-10 membered heterocyclylalkyl, cycloalkylalkyl, optionally substituted phenyl or naphthyl, optionally substituted 5-10 membered heterocyclyl and cycloalkyl;
and pharmaceutically acceptable salts thereof.

The invention also relates to combinations with HGF/SF:c-Met inhibitors including:
N-(4-(6,7-dimethoxyquinolin-4-yloxy)-3-fluorophenyl)-1-methyl-3-oxo-2-phenyl-5-(pyridin-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyrrolidin-1-ylmethyl)-2,3-dihydro-1H-pyrazole-4-carboxamide;
N-(4-(6,7-dimethoxyquinolin-4-yloxy)-3-fluorophenyl)-5-((ethyl(methyl)amino)methyl)-1-methyl-3-oxo-2-phenyl-2,3-dihydro-1H-pyrazole-4-carboxamide;
N-(4-(6,7-dimethoxyquinolin-4-yloxy)-3-fluorophenyl)-5-((dimethylamino)methyl)-1-methyl-3-oxo-2-phenyl-2,3-dihydro-1H-pyrazole-4-carboxamide;
5-(aminomethyl)-N-(3-fluoro-4-(7-methoxyquinolin-4-yloxy)phenyl)-1-methyl-3-oxo-2-phenyl-2,3-dihydro-1H-pyrazole-4-carboxamide;
tert-butyl (4-((3-fluoro-4-(7-methoxyquinolin-4-yloxy)phenyl)carbamoyl)-1-methyl-3-oxo-2-phenyl-2,3-dihydro-1H-pyrazol-5-yl)methylcarbamate;
N-(5-(7-methoxyquinolin-4-yloxy)pyridin-2-yl)-1-methyl-3-oxo-2-phenyl-5-(pyrrolidin-1-ylmethyl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyrrolidin-1-ylmethyl)-2,3-dihydro-1H-pyrazole-4-carboxamide;
N-(3-fluoro-4-(7-methoxyquinolin-4-yloxy)phenyl)-5-methyl-3-oxo-2-phenyl-1-((tetrahydrofuran-2-yl)methyl)-2,3-dihydro-1H-pyrazole-4-carboxamide;
5-((ethyl(methyl)amino)methyl)-N-(3-fluoro-4-(7-methoxyquinolin-4-yloxy)phenyl)-1-methyl-3-oxo-2-phenyl-2,3-dihydro-1H-pyrazole-4-carboxamide;
2-benzyl-N-(5-(7-methoxyquinolin-4-yloxy)pyridin-2-yl)-1-methyl-3-oxo-5-(pyridin-4-yl)-2,3-dihydro-1H-pyrazole-4-carboxamide;
2-benzyl-N-(3-fluoro-4-(7-methoxyquinolin-4-yloxy)phenyl)-1-methyl-3-oxo-5-(pyridin-4-yl)-2,3-dihydro-1H-pyrazole-4-carboxamide;
(S)-N-(5-(7-methoxyquinolin-4-yloxy)pyridin-2-yl)-1-methyl-3-oxo-2-(1-phenylethyl)-5-(pyridin-4-yl)-2,3-dihydro-1H-pyrazole-4-carboxamide;
(S)-N-(3-fluoro-4-(7-methoxyquinolin-4-yloxy)phenyl)-1-methyl-3-oxo-2-(1-phenylethyl)-5-(pyridin-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyridin-4-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyridin-4-yl)-2,3-dihydro-1H-pyrazole-4-carboxamide;
N-(5-(6,7-dimethoxyquinolin-4-yloxy)pyridin-2-yl)-1-methyl-3-oxo-2-phenyl-5-(pyridin-4-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyridin-2-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyridin-2-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(tetrahydro-2H-pyran-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(tetrahydro-2H-pyran-4-yl)-2,3-dihydro-1H-pyrazole-4-carboxamide;
1-methyl-N-(5-((7-(methyloxy)-4-quinolinyl)oxy)-2-pyridinyl)-5-(2-methyl-1,3-thiazol-4-yl)-3-oxo-2-phenyl-2,3-dihydro-1H-pyrazole-4-carboxamide;
N-(5-((6,7-bis(methyloxy)-4-quinolinyl)oxy)-2-pyridinyl)-1-methyl-5-(5-methyl-3-isoxazolyl)-3-oxo-2-phenyl-2,3-dihydro-1H-pyrazole-4-carboxamide;
1-methyl-5-(5-methyl-3-isoxazolyl)-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-1-methyl-5-(5-methyl-3-isoxazolyl)-3-oxo-2-phenyl-2,3-dihydro-1H-pyrazole-4-carboxamide;
1-methyl-N-(5-((7-(methyloxy)-4-quinolinyl)oxy)-2-pyridinyl)-3-oxo-2-phenyl-5-(2-pyrazinyl)-2,3-dihydro-1H-pyrazole-4-carboxamide;
N-(3-fluoro-4-((7-(methyloxy)-4-quinolinyl)oxy)phenyl)-1-methyl-3-oxo-2-phenyl-5-(2-pyrazinyl)-2,3-dihydro-1H-pyrazole-4-carboxamide;
N-(5-((6,7-bis(methyloxy)-4-quinolinyl)oxy)-2-pyridinyl)-1-methyl-3-oxo-2-phenyl-5-(2-pyrazinyl)-2,3-dihydro-1H-pyrazole-4-carboxamide;
N-(5-((6,7-bis(methyloxy)-4-quinolinyl)oxy)-2-pyridinyl)-1-methyl-5-(2-methyl-1,3-thiazol-4-yl)-3-oxo-2-phenyl-2,3-dihydro-1H-pyrazole-4-carboxamide;
N-(3-fluoro-4-((7-(methyloxy)-4-quinolinyl)oxy)phenyl)-1-methyl-5-(2-methyl-1,3-thiazol-4-yl)-3-oxo-2-phenyl-2,3-dihydro-1H-pyrazole-4-carboxamide;
N-(4-(6,7-dimethoxyquinolin-4-yloxy)-3-fluorophenyl)-N,1,5-trimethyl-3-oxo-2-phenyl-2,3-dihydro-1H-pyrazole-4-carboxamide;
2-(3-chlorophenyl)-N-(4-(6,7-dimethoxyquinolin-4-yloxy)-3-fluorophenyl)-1,5-dimethyl-3-oxo-2,3-dihydro-1H-pyrazole-4-carboxamide;
2-(3-chlorophenyl)-N-(5-(6,7-dimethoxyquinolin-4-yloxy)pyridin-2-yl)-1,5-dimethyl-3-oxo-2,3-dihydro-1H-pyrazole-4-carboxamide;
N-(4-(6,7-dimethoxyquinolin-4-yloxy)-3-fluorophenyl)-1,5-dimethyl-3-oxo-2-p-tolyl-2,3-dihydro-1H-pyrazole-4-carboxamide;
N-(4-(6,7-dimethoxyquinolin-4-yloxy)-3-fluorophenyl)-2-(4-fluorophenyl)-1,5-dimethyl-3-oxo-2,3-dihydro-1H-pyrazole-4-carboxamide;
N-(5-(6,7-dimethoxyquinolin-4-yloxy)pyridine-2-yl)-1,5-dimethyl-3-oxo-2-p-tolyl-2,3-dihydro-1H-pyrazole-4-carboxamide;
N-(5-(6,7-dimethoxyquinolin-4-yloxy)pyridin-2-yl)-2-(4-fluorophenyl)-1,5-dimethyl-3-oxo-2,3-dihydro-1H-pyrazole-4-carboxamide;
2-(3-chlorophenyl)-N-(5-(7-methoxyquinolin-4-yloxy)pyridin-2-yl)-1,5-dimethyl-3-oxo-2,3-dihydro-1H-pyrazole-4-carboxamide;
N-(5-(7-methoxyquinolin-4-yloxy)pyridin-2-yl)-1,5-dimethyl-3-oxo-2-p-tolyl-2,3-dihydro-1H-pyrazole-4-carboxamide;
2-(2-chlorophenyl)-N-(5-(6,7-dimethoxyquinolin-4-yloxy)pyridin-2-yl)-1,5-dimethyl-3-oxo-2,3-dihydro-1H-pyrazole-4-carboxamide;
2-(2-chlorophenyl)-N-(4-(6,7-dimethoxyquinolin-4-yloxy)-3-fluorophenyl)-1,5-dimethyl-3-oxo-2,3-dihydro-1H-pyrazole-4-carboxamide;
2-(2-chlorophenyl)-N-(3-fluoro-4-(7-methoxyquinolin-4-yloxy)phenyl)-1,5-dimethyl-3-oxo-2,3-dihydro-1H-pyrazole-4-carboxamide;
N-(3-fluoro-4-(7-methoxyquinolin-4-yloxy)phenyl)-2-(4-fluorophenyl)-1,5-dimethyl-3-oxo-2,3-dihydro-1H-pyrazole-4-carboxamide;
2-(3-chlorophenyl)-N-(3-fluoro-4-(7-methoxyquinolin-4-yloxy)phenyl)-1,5-dimethyl-3-oxo-2,3-dihydro-1H-pyrazole-4-carboxamide;
N-(6-(6,7-dimethoxyquinolin-4-yloxy)pyridin-3-yl)-1,5-dimethyl-3-oxo-2-phenyl-2,3-dihydro-1H-pyrazole-4-carboxamide;
N-(2-chloro-4-(6,7-dimethoxyquinolin-4-yloxy)phenyl)-1,5-dimethyl-3-oxo-2-phenyl-2,3-dihydro-1H-pyrazole-4-carboxamide;
2-benzyl-N-(4-(6,7-dimethoxyquinolin-4-yloxy)-3-fluorophenyl)-1,5-dimethyl-3-oxo-2,3-dihydro-1H-pyrazole-4-carboxamide;
2-benzyl-N-(5-(6,7-dimethoxyquinolin-4-yloxy)pyridin-2-yl)-1,5-dimethyl-3-oxo-2,3-dihydro-1H-pyrazole-4-carboxamide;
N-(5-(7-methoxyquinolin-4-yloxy)pyridin-2-yl)-1-methyl-3-oxo-2-phenyl-2,3-dihydro-1H-pyrazole-4-carboxamide;
N-(4-(6,7-dimethoxyquinolin-4-yloxy)-3-fluorophenyl)-1-(2-hydroxy-2-methylpropyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-3-oxo-1-(2-oxobutyl)-2-phenyl-2,3-dihydro-1H-pyrazole-4-carboxamide;
N-(3-fluoro-4-(7-methoxyquinolin-4-yloxy)phenyl)-5-methyl-1-(3-methyl-2-oxobutyl)-3-oxo-2-phenyl-2,3-dihydro-1H-pyrazole-4-carboxamide;
(R)-N-(3-fluoro-4-(7-methoxyquinolin-4-yloxy)phenyl)-1-(2-hydroxybutyl)-5-methyl-3-oxo-2-phenyl-2,3-dihydro-1H-pyrazole-4-carboxamide;
N-(3-fluoro-4-(7-methoxyquinolin-4-yloxy)phenyl)-1-((2R,3R)-3-hydroxybutan-2-yl)-5-methyl-3-oxo-2-phenyl-2,3-dihydro-1H-pyrazole-4-carboxamide;
1-((2R,3R)-3-hydroxybutan-2-yl)-N-(5-(7-methoxyquinolin-4-yloxy)pyridin-2-yl)-5-methyl-3-oxo-2-phenyl-2,3-dihydro-1H-pyrazole-4-carboxamide;
(S)-1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
(R)-1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
(S)-N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
(R)-N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-1-((3-methyl-2-oxooxazolidin-5-yl)methyl)-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-(methylamino)propyl)-5-methyl-3-oxo-2-phenyl-2,3-dihydro-1H-pyrazole-4-carboxamide;
1-(3-chloro-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-2-methylbutyl)-5-methyl-3-oxo-2-phenyl-2,3-dihydro-1H-pyrazole-4-carboxamide;
1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-morpholinopropyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-1-(oxazolidin-5-ylmethyl)-3-oxo-2-phenyl-2,3-dihydro-1H-pyrazole-4-carboxamide;
(S)-N-(3-fluoro-4-(7-methoxyquinolin-4-yloxy)phenyl)-1-(2-hydroxybutyl)-5-methyl-3-oxo-2-phenyl-2,3-dihydro-1H-pyrazole-4-carboxamide;
1-(3-amino-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-2-methylpropyl)-5-methyl-3-oxo-2-phenyl-2,3-dihydro-1H-pyrazole-4-carboxamide;
(R)-1-(2-hydroxypropyl)-N-(5-(7-methoxyquinolin-4-yloxy)pyridin-2-yl)-5-methyl-3-oxo-2-phenyl-2,3-dihydro-1H-pyrazole-4-carboxamide;
1-(3-(dimethylamino)-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
(R)-N-(3-fluoro-4-(7-methoxyquinolin-4-yloxy)phenyl)-1-(2-hydroxypropyl)-5-methyl-3-oxo-2-phenyl-2,3-dihydro-1H-pyrazole-4-carboxamide;
(R)-N-(4-(6,7-dimethoxyquinolin-4-yloxy)-3-fluorophenyl)-1-(2-hydroxypropyl)-5-methyl-3-oxo-2-phenyl-2,3-dihydro-1H-pyrazole-4-carboxamide;
1-(2-hydroxypropyl)-N-(5-(7-methoxyquinolin-4-yloxy)pyridin-2-yl)-5-methyl-3-oxo-2-phenyl-2,3-dihydro-1H-pyrazole-4-carboxamide;
N-(5-(6,7-dimethoxyquinolin-4-yloxy)pyridin-2-yl)-1-(2-hydroxy-2-methylpropyl)-5-methyl-3-oxo-2-phenyl-2,3-dihydro-1H-pyrazole-4-carboxamide;
(R)-2-(3-chlorophenyl)-N-(3-fluoro-4-(7-methoxyquinolin-4-yloxy)phenyl)-1-(2-hydroxypropyl)-5-methyl-3-oxo-2,3-dihydro-1H-pyrazole-4-carboxamide;
(R)-2-(3-chlorophenyl)-1-(2-hydroxypropyl)-N-(5-(7-methoxyquinolin-4-yloxy)pyridin-2-yl)-5-methyl-3-oxo-2,3-dihydro-1H-pyrazole-4-carboxamide;
(R)-N-(3-fluoro-4-(7-methoxyquinolin-4-yloxy)phenyl)-2-(4-fluorophenyl)-1-(2-hydroxypropyl)-5-methyl-3-oxo-2,3-dihydro-1H-pyrazole-4-carboxamide
1-(2-hydroxy-2-methylpropyl)-N-(5-(1-oxo-7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-hydroxyquinolin-4-yloxy)phenyl)-1-(2-hydroxy-2-methylpropyl)-5-methyl-3-oxo-2-phenyl-2,3-dihydro-1H-pyrazole-4-carboxamide;
1-(2-hydroxy-2-methylpropyl)-N-(5-(7-hydroxyquinolin-4-yloxy)pyridin-2-yl)-5-methyl-3-oxo-2-phenyl-2,3-dihydro-1H-pyrazole-4-carboxamide;
N-(4-(6-ethyl-7-methoxyquinolin-4-yloxy)-3-fluorophenyl)-1,5-dimethyl-3-oxo-2-phenyl-2,3-dihydro-1H-pyrazole-4-carboxamide;
N-(3-fluoro-4-(7-methoxyquinolin-4-yloxy)phenyl)-1,5-dimethyl-3-oxo-2-phenyl-2,3-dihydro-1H-pyrazole-4-carboxamide;
N-(3-fluoro-4-(7-methoxyquinolin-4-yloxy)phenyl)-1,2-dimethyl-3-oxo-5-phenyl-2,3-dihydro-1H-pyrazole-4-carboxamide;
N-(5-(7-methoxyquinolin-4-yloxy)pyridin-2-yl)-1,2-dimethyl-3-oxo-5-phenyl-2,3-dihydro-1H-pyrazole-4-carboxamide;
N-(4-(6,7-dimethoxyquinolin-4-yloxy)-3-fluorophenyl)-1,2-dimethyl-3-oxo-5-phenyl-2,3-dihydro-1H-pyrazole-4-carboxamide;
N-(5-(7-methoxyquinolin-4-yloxy)pyridin-2-yl)-1,5-dimethyl-3-oxo-2-phenyl-2,3-dihydro-1H-pyrazole-4-carboxamide;
(R)-1-(2-hydroxypropyl)-N-(5-(7-methoxyquinolin-4-yloxy)pyridin-2-yl)-2-methyl-3-oxo-5-phenyl-2,3-dihydro-1H-pyrazole-4-carboxamide;
(R)-N-(3-fluoro-4-(7-methoxyquinolin-4-yloxy)phenyl)-1-(2-hydroxypropyl)-2-methyl-3-oxo-5-phenyl-2,3-dihydro-1H-pyrazole-4-carboxamide

(S)-N-(3-fluoro-4-(6-methoxyquinolin-4-yloxy)phenyl)-1-(2-hydroxypropyl)-5-methyl-3-oxo-2-phenyl-2,3-dihydro-1H-pyrazole-4-carboxamide;
1-(2-aminoethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide
1-(2-(1,3-dioxo-1,3-dihydro-2H-isoindol-2-yl)ethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-(2-aminoethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
5-methyl-N-(5-((7-(methyloxy)-4-quinolinyl)oxy)-2-pyridinyl)-3-oxo-2-phenyl-1-(phenylmethyl)-2,3-dihydro-1H-pyrazole-4-carboxamide
1-benzyl-N-(3-fluoro-4-(7-methoxyquinolin-4-yloxy)phenyl)-5-methyl-3-oxo-2-phenyl-2,3-dihydro-1H-pyrazole-4-carboxamide;
5-methyl-1-(2-(methyloxy)ethyl)-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-5-methyl-1-(2-(methyloxy)ethyl)-3-oxo-2-phenyl-2,3-dihydro-1H-pyrazole-4-carboxamide;
1-(2-hydroxyethyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
1-(2R)-2-fluoropropyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
(S)-1-(2-(dimethylamino)propyl)-N-(3-fluoro-4-(7-methoxyquinolin-4-yloxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-3-oxo-2-phenyl-1-(2-(1-pyrrolidinyl)ethyl)-2,3-dihydro-1H-pyrazole-4-carboxamide;
1-((2S)-2-fluoropropyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-1-((2S)-2-fluoropropyl)-5-methyl-3-oxo-2-phenyl-2,3-dihydro-1H-pyrazole-4-carboxamide;
1-((2S)-2-(acetylamino)propyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-((2S)-2-aminopropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-((2S)-2-azidopropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-1-(2-hydroxyethyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-3-oxo-2-phenyl-1-propyl-2,3-dihydro-1H-pyrazole-4-carboxamide;
N-(4-((6,7-bis(methyloxy)-4-quinolinyl)oxy)-3-fluorophenyl)-1-((2R)-2-hydroxypropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-1-((2S)-2-hydroxypropyl)-5-methyl-3-oxo-2-phenyl-2,3-dihydro-1H-pyrazole-4-carboxamide;
5-methyl-N-(5-((7-(methyloxy)-4-quinolinyl)oxy)-2-pyridinyl)-1-(2-methylpropyl)-3-oxo-2-phenyl-2,3-dihydro-1H-pyrazole-4-carboxamide;
5-methyl-N-(5-((7-(methyloxy)-4-quinolinyl)oxy)-2-pyridinyl)-3-oxo-2-phenyl-1-propyl-2,3-dihydro-1H-pyrazole-4-carboxamide;
N-(3-fluoro-4-((7-(methyloxy)-4-quinolinyl)oxy)phenyl)-5-methyl-3-oxo-1-(2-oxopropyl)-2-phenyl-2,3-dihydro-1H-pyrazole-4-carboxamide;
1-(2,3-dihydroxy-2-methylpropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-1-(2-hydroxypropyl)-5-methyl-3-oxo-2-phenyl-2,3-dihydro-1H-pyrazole-4-carboxamide;
N-(4-((6,7-bis(methyloxy)-4-quinazolinyl)oxy)-3-fluorophenyl)-1-(2-hydroxy-2-methylpropyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-1-(2-methyl-2-propen-1-yl)-3-oxo-2-phenyl-2,3-dihydro-1H-pyrazole-4-carboxamide;
N-(4-((6,7-bis(methyloxy)-4-quinolinyl)oxy)-3-fluorophenyl)-1-((2S)-2-hydroxypropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-5-methyl-3-oxo-1-(2-oxopropyl)-2-phenyl-2,3-dihydro-1H-pyrazole-4-carboxamide;
N-(4-((6,7-bis(methyloxy)-4-quinolinyl)oxy)-3-fluorophenyl)-1-(2,3-dihydroxy-2-methylpropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-5-methyl-1-(2-methyl-2-propen-1-yl)-3-oxo-2-phenyl-2,3-dihydro-1H-pyrazole-4-carboxamide;
N-(5-((6,7-bis(methyloxy)-4-quinolinyl)oxy)-2-pyridinyl)-5-methyl-3-oxo-2-phenyl-1-propyl-2,3-dihydro-1H-pyrazole-4-carboxamide;
N-(4-((6,7-bis(methyloxy)-4-quinolinyl)oxy)-3-fluorophenyl)-5-methyl-3-oxo-2-phenyl-2,3-dihydro-1H-pyrazole-4-carboxamide;
N-(5-((6,7-bis(methyloxy)-4-quinolinyl)oxy)-2-pyridinyl)-5-methyl-3-oxo-2-phenyl-1-(2-propen-1-yl)-2,3-dihydro-1H-pyrazole-4-carboxamide;
N-(4-((6,7-bis(methyloxy)-1-oxido-4-quinolinyl)oxy)-3-fluorophenyl)-5-methyl-3-oxo-2-phenyl-1-(2-propen-1-yl)-2,3-dihydro-1H-pyrazole-4-carboxamide;
N-(4-((6,7-bis(methyloxy)-4-quinolinyl)oxy)-3-fluorophenyl)-5-methyl-3-oxo-2-phenyl-1-(phenylmethyl)-2,3-dihydro-1H-pyrazole-4-carboxamide;
4-(6,7-dimethoxyquinolin-4-yloxy)-3-fluoro-N-(5-oxo-1-phenyl-2,5-dihydro-1H-pyrazol-3-yl)benzamide;
4-(6,7-dimethoxyquinolin-4-yloxy)-N-((1,2-dimethyl-5-oxo-3-phenyl-2,5-dihydro-1H-pyrazol-4-yl)methyl)-3-fluorobenzamide;
4-(6,7-dimethoxyquinolin-4-yloxy)-N-(2,3-dimethyl-5-oxo-1-phenyl-2,5-dihydro-1H-pyrazol-4-yl)-3-fluorobenzamide
4-(6,7-dimethoxyquinolin-4-yloxy)-N-((2,3-dimethyl-5-oxo-1-phenyl-2,5-dihydro-1H-pyrazol-4-yl)methyl)-3-fluorobenzamide;
1-benzyl-N-(5-(7-methoxyquinolin-4-yloxy)pyridin-2-yl)-2-oxo-1,2-dihydropyrazolo[1,5-a]pyridine-3-carboxamide;
4-((5-(6,7-dimethoxyquinolin-4-yloxy)pyridin-2-ylamino)methyl)-1,5-dimethyl-2-phenyl-1,2-dihydropyrazol-3-one;
N-(3-fluoro-4-(2-(3-methyl-1,2,4-oxadiazol-5-yl)thieno[3,2-b]pyridin-7-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-((2-(1-methyl-1H-imidazol-5-yl)thieno[3,2-b]pyridin-7-yl)oxy)phenyl)-1-(2-hydroxy-2-methylpropyl)-5-methyl-3-oxo-2-phenyl-2,3-dihydro-1H-pyrazole-4-carboxamide;
N-(3-fluoro-4-((2-(1-methyl-1H-imidazol-5-yl)thieno[3,2-b]pyridin-7-yl)oxy)phenyl)-1-((2R)-2-hydroxypropyl)-5-methyl-3-oxo-2-phenyl-2,3-dihydro-1H-pyrazole-4-carboxamide;
N-(3-fluoro-4-(7H-pyrrolo[2,3-d]pyrimidin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(1H-pyrrolo[2,3-b]pyridin-4-yloxy)phenyl)-1-(2-hydroxy-2-methylpropyl)-5-methyl-3-oxo-2-phenyl-2,3-dihydro-1H-pyrazole-4-carboxamide;
methyl(6-((4-(((1-(2-hydroxy-2-methylpropyl)-5-methyl-3-oxo-2-phenyl-2,3-dihydro-1H-pyrazol-4-yl)carbonyl)amino)phenyl)oxy)-1H-benzimidazol-2-yl)carbamate;
N-(4-(2-(azetidine-1-carbonyl)thieno[3,2-b]pyridin-7-yloxy)-3-fluorophenyl)-5-methyl-3-oxo-2-phenyl-1-propyl-2,3-dihydro-1H-pyrazole-4-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N-methylthieno[3,2-b]pyridine-2-carboxamide;
N-(3-fluoro-4-(2-(1-methylpiperazine-4-carbonyl)thieno[3,2-b]pyridin-7-yloxy)phenyl)-1-(2-hydroxy-2-methylpropyl)-5-methyl-3-oxo-2-phenyl-2,3-dihydro-1H-pyrazole-4-carboxamide;
N-(2-(dimethylamino)ethyl)-7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide;
N-(4-(2-(3-(dimethylamino)pyrrolidine-1-carbonyl)thieno[3,2-b]pyridin-7-yloxy)-3-fluorophenyl)-1-(2-hydroxy-2-methylpropyl)-5-methyl-3-oxo-2-phenyl-2,3-dihydro-1H-pyrazole-4-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N,N-dimethylthieno[3,2-b]pyridine-2-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide;
N-(2-(dimethylamino)ethyl)-7-(2-fluoro-4-(1-(2-hydroxy-2-methylpropyl)-5-methyl-3-oxo-2-phenyl-2,3-dihydro-1H-pyrazole-4-carboxamido)phenoxy)-N-methylthieno[3,2-b]pyridine-2-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N-(2-methoxyethyl)thieno[3,2-b]pyridine-2-carboxamide;
N-(4-(2-(azetidine-1-carbonyl)thieno[3,2-b]pyridin-7-yloxy)-3-fluorophenyl)-1-(2-hydroxy-2-methylpropyl)-5-methyl-3-oxo-2-phenyl-2,3-dihydro-1H-pyrazole-4-carboxamide;
N-cyclopropyl-7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide
7-(2-fluoro-4-(5-methyl-3-oxo-2-phenyl-1-propyl-2,3-dihydro-1H-pyrazole-4-carboxamido)phenoxy)thieno[3,2-b]pyridine-2-carboxamide;
N-(3-fluoro-4-(6-(pyrrolidine-1-carboxamido)pyrimidin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(6-(pyrrolidine-1-carboxamido)pyrimidin-4-yloxy)phenyl)-5-methyl-3-oxo-2-phenyl-1-propyl-2,3-dihydro-1H-pyrazole-4-carboxamide;
N-(6-(4-(1,5-dimethyl-3-oxo-2-phenyl-2,3-dihydro-1H-pyrazole-4-carboxamido)-2-fluorophenoxy)pyrimidin-4-yl)morpholine-4-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)morpholine-4-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)piperidine-1-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)-4-methylpiperazine-1-carboxamide;
(R)-N-(4-(6-(3-(dimethylamino)pyrrolidine-1-carboxamido)pyrimidin-4-yloxy)-3-fluorophenyl)-5-methyl-3-oxo-2-phenyl-1-propyl-2,3-dihydro-1H-pyrazole-4-carboxamide;
(R)-N-(4-(6-aminopyrimidin-4-yloxy)-3-fluorophenyl)-1-(2-hydroxypropyl)-5-methyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-1,5-dimethyl-3-oxo-2-phenyl-2,3-dihydro-1H-pyrazole-4-carboxamide;
N-(4-(4-(1,5-dimethyl-3-oxo-2-phenyl-2,3-dihydro-1H-pyrazole-4-carboxamido)-2-fluorophenoxy)pyridin-2-yl)piperidine-1-carboxamide;
(R)-N-(4-(2-(3-(dimethylamino)pyrrolidine-1-carboxamido)pyridin-4-yloxy)-3-fluorophenyl)-1,5-dimethyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-5-methyl-3-oxo-2-phenyl-1-propyl-2,3-dihydro-1H-pyrazole-4-carboxamide;
N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide;
N-(4-(2-fluoro-4-(1-(2-hydroxy-2-methylpropyl)-5-methyl-3-oxo-2-phenyl-2,3-dihydro-1H-pyrazole-4-carboxamido)phenoxy)pyridin-2-yl)piperidine-1-carboxamide;
5-methyl-N-(4-((7-(methyloxy)-4-quinolinyl)methyl)phenyl)-3-oxo-2-phenyl-1-propyl-2,3-dihydro-1H-pyrazole-4-carboxamide;
N-(4-(hydroxy(7-methoxyquinolin-4-yl)methyl)phenyl)-5-methyl-3-oxo-2-phenyl-1-propyl-2,3-dihydro-1H-pyrazole-4-carboxamide;
1,5-dimethyl-N-(5-((7-(methyloxy)-4-quinolinyl)oxy)-2-pyrimidinyl)-3-oxo-2-phenyl-2,3-dihydro-1H-pyrazole-4-carboxamide;
5-methyl-N-(4-((7-(methyloxy)-4-quinolinyl)sulfinyl)phenyl)-3-oxo-2-phenyl-1-propyl-2,3-dihydro-1H-pyrazole-4-carboxamide
1-(2-hydroxy-2-methylpropyl)-5-methyl-N-(4-((7-(methyloxy)-4-quinolinyl)thio)phenyl)-3-oxo-2-phenyl-2,3-dihydro-1H-pyrazole-4-carboxamide
5-methyl-N-(4-((7-(methyloxy)-4-quinolinyl)thio)phenyl)-3-oxo-2-phenyl-1-propyl-2,3-dihydro-1H-pyrazole-4-carboxamide
5-methyl-N-(3-((7-(methyloxy)-4-quinolinyl)oxy)propyl)-3-oxo-2-phenyl-1-propyl-2,3-dihydro-1H-pyrazole-4-carboxamide;
5-methyl-N-(trans-4-((7-(methyloxy)-4-quinolinyl)oxy)cyclohexyl)-3-oxo-2-phenyl-1-propyl-2,3-dihydro-1H-pyrazole-4-carboxamide;
5-methyl-N-(cis-4-((7-(methyloxy)-4-quinolinyl)oxy)cyclohexyl)-3-oxo-2-phenyl-1-propyl-2,3-dihydro-1H-pyrazole-4-carboxamide;
1-(2-hydroxy-2-methylpropyl)-5-methyl-N-(trans-4-((7-(methyloxy)-4-quinolinyl)oxy)cyclohexyl)-3-oxo-2-phenyl-2,3-dihydro-1H-pyrazole-4-carboxamide;
5-methyl-N-(4-((7-(methyloxy)-4-quinolinyl)amino)phenyl)-3-oxo-2-phenyl-1-propyl-2,3-dihydro-1H-pyrazole-4-carboxamide;
5-methyl-N-(5-((7-(methyloxy)-4-quinolinyl)oxy)-2-pyrimidinyl)-3-oxo-2-phenyl-1-propyl-2,3-dihydro-1H-pyrazole-4-carboxamide;
N-(3-fluoro-4-((7-(methyloxy)-4-quinolinyl)amino)phenyl)-1-(2-hydroxy-2-methylpropyl)-5-methyl-3-oxo-2-phenyl-2,3-dihydro-1H-pyrazole-4-carboxamide;
1-(2-hydroxy-2-methylpropyl)-5-methyl-4-((7-((7-(methyloxy)-4-quinolinyl)oxy)-2,3-dihydro-4H-1,4-benzoxazin-4-yl)carbonyl)-2-phenyl-1,2-dihydro-3H-pyrazol-3-one;
1-(2-hydroxy-2-methylpropyl)-5-methyl-N-(4-((7-(methyloxy)-4-quinolinyl)amino)phenyl)-3-oxo-2-phenyl-2,3-dihydro-1H-pyrazole-4-carboxamide;
N-(4-(6,7-dimethoxyquinolin-4-yloxy)-3-fluorophenyl)-3-hydroxy-2-(1-oxoisoindolin-2-yl)propanamide;
N-(4-(6,7-dimethoxyquinolin-4-yloxy)-3-fluorophenyl)-2-(1-oxoisoindolin-2-yl)acetamide;
N-(4-(6,7-dimethoxyquinolin-4-yloxy)-3-fluorophenyl)-2-oxo-1,5-diphenyl-1,2-dihydropyridine-3-carboxamide;
N-(5-((6,7-bis(methyloxy)-4-quinolinyl)oxy)-2-pyridinyl)-6-oxo-1-(phenylmethyl)-1,1′,2′,3′,6,6′-hexahydro-3,4′-bipyridine-5-carboxamide;
N-(5-((6,7-bis(methyloxy)-4-quinolinyl)oxy)-2-pyridinyl)-6-oxo-1-(phenylmethyl)-1,6-dihydro-3,3′-bipyridine-5-carboxamide;
N-(5-((6,7-bis(methyloxy)-4-quinolinyl)oxy)-2-pyridinyl)-6′-oxo-1′-(phenylmethyl)-1′,6′-dihydro-2,3′-bipyridine-5′-carboxamide
N-(5-((6,7-bis(methyloxy)-4-quinolinyl)oxy)-2-pyridinyl)-2-oxo-1-(phenylmethyl)-5-(2-thienyl)-1,2-dihydro-3-pyridinecarboxamide;
N-(5-((6,7-bis(methyloxy)-4-quinolinyl)oxy)-2-pyridinyl)-2-oxo-1-(phenylmethyl)-5-(2-pyrazinyl)-1,2-dihydro-3-pyridinecarboxamide;
N-(5-((6,7-bis(methyloxy)-4-quinolinyl)oxy)-2-pyridinyl)-5-methyl-2-oxo-1-(phenylmethyl)-1,2-dihydro-3-pyridinecarboxamide;
N-(4-((6,7-bis(methyloxy)-4-quinolinyl)oxy)-3-fluorophenyl)-5-bromo-1-(3-methylphenyl)-2-oxo-1,2-dihydro-3-pyridinecarboxamide;
N-(4-((6,7-bis(methyloxy)-4-quinolinyl)oxy)-3-fluorophenyl)-5-(1-methyl-1H-pyrazol-4-yl)-2-oxo-1-phenyl-1,2-dihydro-3-pyridinecarboxamide;
N-(3-fluoro-4-((6-(methyloxy)-7-((3-(4-morpholinyl)propyl)oxy)-4-quinolinyl)oxy)phenyl)-2-oxo-5-phenyl-1-(phenylmethyl)-1,2-dihydro-3-pyridinecarboxamide;
1,1-dimethylethyl 5-(((5-((6,7-bis(methyloxy)-4-quinolinyl)oxy)-2-pyridinyl)amino)carbonyl)-6-oxo-1-(phenylmethyl)-1,3′,6,6′-tetrahydro-3,4′-bipyridine-1′(2′H)-carboxylate;
N-(4-((6,7-bis(methyloxy)-4-quinolinyl)oxy)-3-fluorophenyl)-2-oxo-1-(phenylmethyl)-5-(2-pyrimidinyl)-1,2-dihydro-3-pyridinecarboxamide;
N-(4-((6,7-bis(methyloxy)-4-quinolinyl)oxy)-3-fluorophenyl)-2-oxo-1-phenyl-5-(1H-pyrazol-4-yl)-1,2-dihydro-3-pyridinecarboxamide;
1-benzyl-5-bromo-N-(2-chloro-4-(6,7-dimethoxyquinolin-4-yloxy)phenyl)-2-oxo-1,2-dihydropyridine-3-carboxamide;
N-(5-(7-methoxyquinolin-4-yloxy)pyridin-2-yl)-2-oxo-1-phenyl-5-(pyridin-3-yl)-1,2-dihydropyridine-3-carboxamide;
N-(5-(7-methoxyquinolin-4-yloxy)pyridin-2-yl)-2-oxo-1-phenyl-5-(pyrazin-2-yl)-1,2-dihydropyridine-3-carboxamide;
N-(5-(6,7-dimethoxyquinolin-4-yloxy)pyridin-2-yl)-2-oxo-1-phenyl-5-(pyridin-3-yl)-1,2-dihydropyridine-3-carboxamide;
N-(5-(6,7-dimethoxyquinolin-4-yloxy)pyridin-2-yl)-2-oxo-1-phenyl-5-(pyrazin-2-yl)-1,2-dihydropyridine-3-carboxamide;
N-(5-(6,7-dimethoxyquinolin-4-yloxy)pyridin-2-yl)-2-oxo-1-phenyl-5-(thiophen-2-yl)-1,2-dihydropyridine-3-carboxamide;
5-benzyl-N-(5-(6,7-dimethoxyquinolin-4-yloxy)pyridin-2-yl)-2-oxo-1-phenyl-1,2-dihydropyridine-3-carboxamide;
tert-butyl 4-(5-((5-(6,7-dimethoxyquinolin-4-yloxy)pyridin-2-yl)carbamoyl)-6-oxo-1-phenyl-1,6-dihydropyridin-3-yl)-5,6-dihydropyridine-1(2H)-carboxylate;
5-bromo-N-(2-chloro-4-(6,7-dimethoxyquinolin-4-yloxy)phenyl)-2-oxo-1-phenyl-1,2-dihydropyridine-3-carboxamide;
N-(5-(6,7-dimethoxyquinolin-4-yloxy)pyridin-2-yl)-4-(2-methoxyethylamino)-2-oxo-1-phenyl-1,2-dihydropyridine-3-carboxamide;
N-(5-(6,7-dimethoxyquinolin-4-yloxy)pyridin-2-yl)-2-oxo-1-phenyl-4-(tetrahydro-2H-pyran-4-ylamino)-1,2-dihydropyridine-3-carboxamide;
N-(5-(6,7-dimethoxyquinolin-4-yloxy)pyridin-2-yl)-2-oxo-1-phenyl-4-(phenylamino)-1,2-dihydropyridine-3-carboxamide;
N-(5-(6,7-dimethoxyquinolin-4-yloxy)pyridin-2-yl)-4-(4-methylpiperazin-1-yl)-2-oxo-1-phenyl-1,2-dihydropyridine-3-carboxamide;
N-(5-(6,7-dimethoxyquinolin-4-yloxy)pyridin-2-yl)-4-(methylamino)-2-oxo-1-phenyl-1,2-dihydropyridine-3-carboxamide;
N-(5-(6,7-dimethoxyquinolin-4-yloxy)pyridin-2-yl)-4-(dimethylamino)-2-oxo-1-phenyl-1,2-dihydropyridine-3-carboxamide;
4-(2-methoxyethylamino)-N-(5-(7-methoxyquinolin-4-yloxy)pyridin-2-yl)-2-oxo-1-phenyl-1,2-dihydropyridine-3-carboxamide;
N-(3-fluoro-4-(7-methoxyquinolin-4-yloxy)phenyl)-4-(2-methoxyethylamino)-2-oxo-1-phenyl-1,2-dihydropyridine-3-carboxamide;
N-(4-((6,7-bis(methyloxy)-4-quinolinyl)oxy)-3-fluorophenyl)-1-cyclopentyl-6-oxo-5-(2-oxo-1-pyrrolidinyl)-1,6-dihydro-3-pyridinecarboxamide;
1-benzyl-N-(5-(6,7-dimethoxyquinolin-4-yloxy)pyridin-2-yl)-4-(2-methoxyethylamino)-2-oxo-1,2-dihydropyridine-3-carboxamide;
1-benzyl-N-(5-(6,7-dimethoxyquinolin-4-yloxy)pyridin-2-yl)-4-(dimethylamino)-2-oxo-1,2-dihydropyridine-3-carboxamide;
1-benzyl-N-(5-(6,7-dimethoxyquinolin-4-yloxy)pyridin-2-yl)-4-(methylamino)-2-oxo-1,2-dihydropyridine-3-carboxamide;
1-benzyl-N-(5-(6,7-dimethoxyquinolin-4-yloxy)pyridin-2-yl)-2-oxo-4-(phenylamino)-1,2-dihydropyridine-3-carboxamide;
1-benzyl-N-(5-(6,7-dimethoxyquinolin-4-yloxy)pyridin-2-yl)-2-oxo-4-(pyridin-4-ylamino)-1,2-dihydropyridine-3-carboxamide;
1-benzyl-N-(5-(6,7-dimethoxyquinolin-4-yloxy)pyridin-2-yl)-4-(4-methylpiperazin-1-yl)-2-oxo-1,2-dihydropyridine-3-carboxamide;
1-benzyl-N-(5-(6,7-dimethoxyquinolin-4-yloxy)pyridin-2-yl)-2-oxo-4-(tetrahydro-2H-pyran-4-ylamino)-1,2-dihydropyridine-3-carboxamide;
1-benzyl-N-(5-(6,7-dimethoxyquinolin-4-yloxy)pyridin-2-yl)-2-oxo-4-(4-(trifluoromethyl)phenylamino)-1,2-dihydropyridine-3-carboxamide;
1-cyclopentyl-N-(4-(6,7-dimethoxyquinolin-4-yloxy)-3-fluorophenyl)-6-oxo-5-(2-oxopyrrolidin-1-yl)-1,6-dihydropyridine-3-carboxamide;
N-(3-fluoro-4-(2-(pyrrolidine-1-carboxamido)pyridin-4-yloxy)phenyl)-3-oxo-2-phenyl-2,3-dihydropyridazine-4-carboxamide;
6-((diethylamino)methyl)-N-(4-(6,7-dimethoxyquinolin-4-yloxy)-3-fluorophenyl)-3-oxo-2-phenyl-2,3-dihydropyridazine-4-carboxamide;
6-((dimethylamino)methyl)-N-(3-fluoro-4-(7-methoxyquinolin-4-yloxy)phenyl)-3-oxo-2-phenyl-2,3-dihydropyridazine-4-carboxamide;
N-(3-fluoro-4-(7-methoxyquinolin-4-yloxy)phenyl)-6-methyl-3-oxo-2-phenyl-2,3-dihydropyridazine-4-carboxamide;
N-(5-(6,7-dimethoxyquinolin-4-yloxy)pyridin-2-yl)-6-methyl-3-oxo-2-phenyl-2,3-dihydropyridazine-4-carboxamide;
2-benzyl-N-(5-(6,7-dimethoxyquinolin-4-yloxy)pyridin-2-yl)-6-methyl-3-oxo-2,3-dihydropyridazine-4-carboxamide;
N-(3-fluoro-4-(7-methoxyquinolin-4-yloxy)phenyl)-3-oxo-2-phenyl-2,3-dihydropyridazine-4-carboxamide;
N-(2-chloro-4-(6,7-dimethoxyquinolin-4-yloxy)phenyl)-6-methyl-3-oxo-2-phenyl-2,3-dihydropyridazine-4-carboxamide;
(R)-N-(4-(6,7-dimethoxyquinolin-4-yloxy)-3-fluorophenyl)-643-(dimethylamino)pyrrolidin-1-yl)methyl)-3-oxo-2-phenyl-2,3-dihydropyridazine-4-carboxamide;
3-benzyl-N-(4-(6,7-dimethoxyquinolin-4-yloxy)-3-fluorophenyl)-2-oxoimidazolidine-1-carboxamide;
N-(4-(6,7-dimethoxyquinolin-4-yloxy)-3-fluorophenyl)-5-((dimethylamino)methyl)-2-oxo-3-phenyl-tetrahydropyrimidine-1(2H)-carboxamide;
N-(3-fluoro-4-(7-methoxyquinolin-4-yloxy)phenyl)-3-oxo-4-phenylmorpholine-2-carboxamide;
N-(5-(7-methoxyquinolin-4-yloxy)pyridin-2-yl)-1-methyl-3-oxo-2-phenyl-2,3-dihydro-1H-pyrazole-4-carboxamide; and
N-(3-fluoro-4-(7-methoxyquinolin-4-yloxy)phenyl)-3-oxo-4-phenylmorpholine-2-carboxamide.

The invention also relates to combinations with HGF/SF:c-Met inhibitors, 1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (Amgen Compound 2) and/or N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide (Amgen Compound 3).

The invention also relates to combinations with HGF/SF:c-Met inhibitors including ARQ197, MK2461, MK 8033, PF04217903, PF2341066, JNJ38877605, XL880, XL184, MGCD265, BMS 777607 and E7050.

The invention also relates to combinations with HGF/SF:c-Met inhibitors that are antibodies or antigen binding fragments (e.g., antibodies or antigen binding fragments that bind to HGF/SF and/or c-Met) (“HGF/SF:c-Met antibodies”).

The invention also relates to combinations with monoclonal HGF/SF:c-Met antibodies and Fab fragments of HGF/SF:c-Met monoclonal antibodies, such as those described in U.S. Pat. No. 5,646,036 and U.S. Pat. No. 5,686,292.

The invention also relates to combinations with humanized or fully human HGF/SF:c-Met antibodies, such as those described in US 2005/0118643, WO 2005/017107, US 2007/0092520, WO 2005/107800, WO 2007/115049, and U.S. Pat. No. 7,494,650 and U.S. Pat. No. 7,220,410.

The invention also relates to combinations with L2G7 and/or OA-5d5 and/or AMG 102.

The invention also relates to a kit comprising, in one or more containers, separately or in admixture one or more HGF/SF:c-Met inhibitors and one or more VEGF inhibitors in accordance with any of the foregoing.

The invention also relates to a kit, wherein the inhibitors are comprised in pharmaceutically acceptable formulations.

The invention also relates to a kit, comprising motesanib and AMG 102 and/or Amgen Compound 2 and/or Amgen Compound 3.

The invention also relates to a kit, wherein the inhibitors are disposed in separate containers.

The invention also relates to a kit according to any of the foregoing, further comprising integrally thereto or as one or more separate documents, information pertaining to the contents or the kit and the use of the inhibitors.

The invention also relates to a kit according to any of the foregoing, wherein the compositions are formulated for reconstitution in a diluent.

The invention also relates to a kit according to any of the foregoing, further comprising a container of sterile diluent.

The invention also relates to a kit according to any of the foregoing, wherein said compositions are disposed in vials under partial vacuum sealed by a septum and suitable for reconstitution to form a formulation effective for parental administration.

As used in relation to the invention, the term “treating” or “treatment” and the like should be taken broadly. They should not be taken to imply that a subject is treated to total recovery. Accordingly, these terms include amelioration of the symptoms or severity of a particular condition or preventing or otherwise reducing the risk of further development of a particular condition.

The term “comprising” is meant to be open ended, including the indicated component but not excluding other elements.

The phrase “therapeutically-effective” is intended to qualify the amount of each agent, which will achieve the goal of improvement in disorder severity and the frequency of incidence over treatment of each agent by itself, while avoiding adverse side effects typically associated with alternative therapies. For example, effective neoplastic therapeutic agents prolong the survivability of the patient, inhibit the rapidly-proliferating cell growth associated with the neoplasm, or effect a regression of the neoplasm.

It should be appreciated that methods of the invention may be applicable to various species of subjects, preferably mammals, more preferably humans.

As used herein, the compounds of the present invention include the pharmaceutically acceptable derivatives thereof.

Where the plural form is used for compounds, salts, and the like, this is taken to mean also a single compound, salt and the like.

The terms “combination” and “cotherapy” are used interchangeably herein. The terms “combination” and “cotherapy” refer herein to the administration of a single formulation comprising at least two active agents, as well as sequential administration of at least two active agents or formulations thereof.

The terms “cancer” and “cancerous” when used herein refer to or describe the physiological condition in mammals that is typically characterized by unregulated cell growth. Examples of cancer include but are not limited to, carcinoma, lymphoma, sarcoma, blastoma and leukemia. More particular examples of such cancers include squamous cell carcinoma, lung cancer, including non-small cell lung cancer, pancreatic cancer, cervical cancer, bladder cancer, hepatoma, breast cancer, colon carcinoma, including colorectal cancer, kidney cancer, including renal cell carcinoma and head and neck cancer, including Glioblastoma Multiforme (GBM).

A VEGFR inhibitor is defined as a compound that inhibits the receptor as shown with in vitro testing or by other means.

The following are among specific VEGF inhibitors that may be used in the invention in this regard:

AEE-788 (Novartis) (also called AE-788 and NVP-AEE-788, among others) including formulations for oral administration and closely related VEGF inhibitors;

AG-13736 (Pfizer) (axitinib) (also called AG-013736) including formulations for oral administration and closely related VEGF inhibitors;

AG-028262 (Pfizer) and closely related VEGF inhibitors;

AVE-8062 (Ajinomoto Co. and Sanofi-aventis) (also called AC-7700 and combretastatin A4 analog, among others), and closely related VEGF inhibitors;

AZD-2171 (AstraZeneca) (cediranib) (also called AZ-2171) and closely related VEGF inhibitors;

NEXAVAR® (sorafenib)) (Bayer AG and Onyx) (also called CAS Registry Number 284461-73-0, BAY-43-9006, raf kinase inhibitor, sorafenib, sorafenib analogs, and IDDBCP150446, among others) and closely related VEGF inhibitors;

BMS-387032 (Sunesis and Bristol-Myers Squibb) (also called SNS-032 and CAS Registry Number 345627-80-7, among others) and closely related VEGF inhibitors;

CEP-7055 (Cephalon and Sanofi-aventis) (also called CEP-11981 and SSR-106462, among others) and closely related VEGF inhibitors; CHIR-258 (Chiron) (also called CAS Registry Number 405169-16-6, GFKI, and GFKI-258, among others) and closely related VEGF inhibitors;

CP-547632 (OSI Pharmaceuticals and Pfizer) (also called CAS Registry Number 252003-65-9, among others) and closely related VEGF inhibitors such as, for instance, CP-564959;

E-7080 (Eisai Co.) (also called CAS Registry Number 417716-92-8 and ER-203492-00, among others) and closely related VEGF inhibitors;

Pazopanib (GlaxoSmithKline) and closely related VEGF inhibitors;

GW-654652 (GlaxoSmithKline) and closely related indazolylpyrimidine Kdr inhibitors;

KRN-951 (Kirin Brewery Co.) and other closely related quinoline-urea VEGF inhibitors;

PKC-412 (Novartis) (also called CAS Registry Number 120685-11-2, benzoylstaurosporine, CGP-41251, midostaurin, and STI-412, among others) and closely related VEGF inhibitors;

PTK-787 (Novartis and Schering) (also called CAS Registry Numbers 212141-54-3 and 212142-18-2, PTK/ZK, PTK-787/ZK-222584, ZK-22584, VEGF-TKI, VEGF-RKI, PTK-787A, DE-00268, CGP-79787, CGP-79787D, vatalanib, ZK-222584, among others) and closely related anilinophthalazine derivative VEGF inhibitors;

SU11248 (Sugen and Pfizer) (also called SU-11248, SU-011248, SU-11248J, SUTENT®, and sunitinib malate, among others) and closely related VEGF inhibitors;

SU-5416 (Sugen and Pfizer/Pharmacia) (also called CAS Registry Number 194413-58-6, semaxanib, 204005-46-9, among others) and closely related VEGF inhibitors;

SU-6668 (Sugen and Taiho) (also called CAS Registry Number 252916-29-3, SU-006668, and TSU-68, among others) and closely related VEGF inhibitors as described in, among others, WO 99/48868, WO 99/61422, and WO 00/038519, which are hereby incorporated by reference in their entireties, particularly in parts pertaining to SU-6668 and closely related VEGF inhibitors, their structures and properties, and methods for making and using them;

Thalidomide (Celgene) (also called CAS Registry Number 50-35-1, Synovir, Thalidomide Pharmion, and Thalomid, among others) and closely related VEGF inhibitors;

XL-647 (Exelixis) (also called EXEL-7647, among others) and closely related VEGF inhibitors;

XL-999 (Exelixis) (also called EXEL-0999, among others) and closely related VEGF inhibitors;

ZD-6474 (AstraZeneca) (also called CAS Registry Number 443913-73-3, Zactima, and AZD-6474, among others) and closely related anilinoquinazoline VEGF inhibitors; and

ZK-304709 (Schering) (also called CDK inhibitors (indirubin derivatives), ZK-CDK, MTGI, and multi-target tumor growth inhibitor, among others) and other closely related compounds including the indirubin derivative VEGF inhibitors described in WO 00/234717, WO 02/074742, WO 02/100401, WO 00/244148, WO 02/096888, WO 03/029223, WO 02/092079, and WO 02/094814 which are hereby incorporated by reference in their entireties particularly in parts pertinent to these and closely related VEGF inhibitors, their structures and properties, and methods for making and using them.

Also among VEGF inhibitors in this regard are: Pazopanib, CDP791, Enzastaurin, Boehringer Ingelheim BIBF 1120, BAY 573952, BAY 734506, XL 184, IMC-1121B, CEP 701, SU 014813, SU 10944, SU 12662, OSI-930, and BMS 582664, and closely related VEGF inhibitors.

In addition to the foregoing inhibitors that act directly on VEGF or VEGFR, the following inhibitors have anti-angiogenic properties and can be used in the invention in much the same way as inhibitors that act directly:

ZD-6126 (AstraZeneca and Angiogene) (also called CAS Registry Number 219923-05-4, N-acetylcolchinol phosphate, ANG-453, AZD-6126, ZD-6126 derivatives and ZM-445526, among others) and closely related VEGF inhibitors such as other inhibitors in the ANG-400 series;

Imatinib (Novartis) (also called CAS Registry Numbers 152459-95-5 and 220127-57-1, Glivec, Gleevec, STI-571, and CGP-57148, among others) and closely related VEGF inhibitors;

RAD-001 (Novartis) (also called CAS Registry Number 159351-69-6, RAD-001, SDZ-RAD, Certican, and everolimus, among others) and closely related VEGF inhibitors; and

BMS-354825 (Bristol-Myers Squibb) (also called CAS Registry Number 302962-49-8, Src/Abl kinase inhibitor, and dasatinib, among others) and closely related VEGF inhibitors.

Also useful in the invention in this are regard are CCI-779, 17-AAG, DMXAA, CI-1040, and CI-1033.

Among the VEGF inhibitors contemplated in the invention are the following: (a) a compound described in US 2003/0125339 which is herein incorporated by reference in its entirety, particularly in parts disclosing VEGF inhibitors; (b) a substituted alkylamine derivative described in US 2003/0125339 or US 2003/0225106 each of which is herein incorporated by reference in its entirety, particularly in parts disclosing VEGF inhibitors; (c) a substituted omega-carboxyaryl diphenyl urea or derivative thereof as described in WO 00/42012, WO 00/41698, US 2005/0038080A1, US 2003/0125359A1, US 2002/0165394A1, US 2001/003447A1, US 2001/0016659A1, and US 2002/013774A1 which are herein incorporated by reference in their entirety, particularly in parts disclosing the foregoing VEGF inhibitors; (d) an anilinophthalazine or derivative thereof that binds to and inhibits the activity of multiple receptor tyrosine kinases including binding to the protein kinase domain and inhibition of VEGFR1 and VEGFR2; and (e) (5-[5-fluoro-2-oxo-1,2-dihydroindol-(3Z)-ylidenemethyl]-2,4-dimethyl-1H-pyrrole-3-carboxylic acid [2-diethylaminoethyl]amide) or derivative thereof that are VEGF inhibitors.

In this regard, certain of the VEGF inhibitors are further described below,

(1) motesanib;

(2) NEXAVAR;

(3) AZD-2171;

(4) AG-13736;

(5) AVASTIN;

(6) PTK/ZK; and

(7) SUTENT.

Among these, motesanib is among the contemplated VEGF inhibitors.

“Nexavar®” (also known as BAY 43-9006, sorafenib, CAS Registry Number 284461-73-0, raf kinase inhibitor, sorafenib analogs, and IDDBCP150446, among others) is a substituted omega carboxy diphenyl urea that inhibits RAF-1 activation, and thereby decreases RAF-1 dependent phosphorylation of MEK-1 and ERK-1, as described in US Patent Application No. 2003/0125359A1, WO 03/047523A2, and Wilhelm et al., Current Pharmaceutical Design, 8:2255-2257 (2002), each of which is herein incorporated by reference in its entirety, particularly in parts pertinent to Nexavar®, its structure and properties, methods for making and using it, and other related molecules. Its chemical name is 4-(4-{3-[4-chloro-3-(trifluoromethyl)phenyl]ureido}phenoxy)-N²-methylpyridine-2-carboxamide. A variety of derivatives have been produced. Among these are fluorinated derivatives described in US Patent Application 2005/0038080A1 and WO 2005/009961A2, which are herein incorporated by reference in their entireties, particularly as to these and other pharmaceutically active diphenyl urea compounds

“PTK/ZK,” also known as vatalanib, is a multi-VEGF receptor tyrosine kinase inhibitor that is said to block tumor angiogenesis and lymphangiogenesis. Its chemical name is N-(4-chlorophenyl)-4-(pyridin-4-ylmethyl)phthalazin-1-amine. It also is known as CAS Registry Numbers 212141-54-3 and 212142-18-2, PTK787, PTK787/ZK, PTK-787/ZK-222584, PTK787/ZK222584, ZK-22584, VEGF-TKI, VEGF-RKI, PTK-787A, DE-00268, CGP-79787, CGP-79787D, vatalanib, and ZK-222584. See Thomas, A., et al., J. of Clin. Oncology, 23(18): 4162-4171 (2005); US Patent Application 2005/0118600A1, which are herein incorporated by reference in their entirety, particularly as to the structure, synthesis, properties, and uses of PTK/ZK and related compounds.

“Sutent®” is a small molecule receptor tyrosine kinase inhibitor with the chemical name (5-[5-fluoro-2-oxo-1,2-dihydroindol-(3Z)-ylidenemethyl]-2,4-dimethyl-1H-pyrrole-3-carboxylic acid [2-diethylaminoethyl]amide). Sutent® is also known as sunitinib malate, SU11248, SU-11248, SU-011248, and SU-11248J, and is reported to have anti-angiogenic and anti-tumor activities. See Mendel, D., et al., Clinical Cancer Research, 9:327-337 (2003); Schlessinger, J., The Scientist, 19(7):17 (2005), which are herein incorporated by reference in their entirety, particularly as to the structure, synthesis, properties, and uses of Sutent® and related compounds.

“Avastin®,” also known as bevacizumab, is a recombinant humanized antibody to VEGF that binds to and inhibits VEGF.

“Motesanib” (AMG 706) is a multi-kinase inhibitor that interferes with the Kit, Ret, PDGF, and VEGF-signalling pathways, as described in U.S. Pat. No. 6,995,162, which is herein, incorporated by reference in its entirety, particularly in parts pertinent to motesanib, its structure and properties, methods for making and using it, and other related compounds. Its chemical name is N-(2,3-dihydro-3,3-dimethyl-1H-indol-6-yl)-2-[(4-pyridinylmethyl)amino]-3-pyridinecarboxamide. As used herein the term motesanib includes pharmaceutically acceptable salts, in particular, the diphosphate salt, except as otherwise provided herein.

An HGF/SF:c-Met inhibitor is defined as any small molecule (i.e., a compound with a molecular weight less than about 1000) or large molecule (i.e., a protein such as an antibody or antigen binding fragment) that interferes with the binding between HGF/SF and c-Met or otherwise blocks the kinase activity of c-Met, as shown with in vitro testing or by other means.

A c-Met inhibitor is defined as a small molecule or large molecule that inhibits the c-Met receptor, as shown with in vitro testing or by other means.

The following are among specific c-Met inhibitors that are contemplated in the invention:

Amgen Compound 2 (1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide) is a selective c-Met inhibitor, as described in WO 2006/116713, which is herein incorporated by reference in its entirety, particularly in parts pertinent to Amgen Compound 2 as it relates to its structure and properties, methods for making and using them, and other related compounds, including pharmaceutically acceptable salts.

Amgen Compound 3 (N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide) is a selective c-Met inhibitor, as described in WO 2006/116713, which is herein incorporated by reference in its entirety, particularly in parts pertinent to Amgen Compound 3, its structure and properties, methods for making and using

XL880 (Exelixis) (also called EXEL-2880 and GSK1363089, among others), a multi-kinase inhibitor that interferes with c-Met pathways, including formulation for oral administration and closely related c-Met inhibitors;

XL184 (Exelixis) including formulations for oral administration and closely related c-Met inhibitors;

PF-2341066 (Pfizer) including formulations for oral administration and closely related c-Met inhibitors;

PF04217903 (Pfizer) including formulations for oral administration and closely related c-Met inhibitors;

ARQ197 (ArQule) including formulations for oral administration and closely related c-Met inhibitors;

MK2461 (Merck) including formulations for oral administration and closely related c-Met inhibitors;

MK8033 (Merck) including formulations for oral administration and closely related c-Met inhibitors;

ARQ 197 (ArQule) including formulations for oral administration and closely related c-Met inhibitors;

MGCD265 (Methylgene) including formulations for oral administration and closely related c-Met inhibitors;

JNJ38877605 (Johnson & Johnson) including formulations for oral administration and closely related c-Met inhibitors;

BMS 777607 (Bristol Myers Squibb) including formulations for oral administration and closely related c-Met inhibitors;

E7050 (Eisai) including formulations for oral administration and closely related c-Met inhibitors;

MP-470 (SuperGen) including formulations for oral administration and closely related c-Met inhibitors;

Compound X (N-[4-(6,7-dimethoxyquinolin-4-yloxy)-3-fluorophenyl]-N-phenylactylthiourea), as claimed in US 2004/0242603, which is herein incorporated by reference in its entirety, particularly in parts pertinent to its structure and properties, methods for making and using it, and other related compounds. Compound X includes pharmaceutically acceptable salts, as well as formulations for oral administration and closely related c-Met inhibitors; and

OA-5d5 (Genentech) (also called One Armed 5d5, 5d5, MetMab, PRO143966, among others) including formulations for oral administration and closely related c-Met inhibitors. OA-5d5 is a humanized anti-c-Met antibody, as described in US 2007/0092520, which is herein incorporated by reference in its entirety, particularly in parts pertinent to OA-5d5, its structure and properties, methods for making and using it, and other related compounds.

Among the foregoing c-Met inhibitors, Amgen Compound 2 and Amgen Compound 3 are contemplated.

An HGF/SF inhibitor is defined as a small molecule or large molecule that interferes with the binding between HGF/SF and c-Met by binding to and neutralizing HGF/SF, as shown with in vitro testing or by other means.

An anti-HGF/SF antibody is defined as an antibody, or fragment thereof, that interferes with the binding between HGF/SF and c-Met by binding to and neutralizing HGF/SF, as shown with in vitro testing or by other means, such as AMG 102 or L2G7 (Takeda-Galaxy Biotech).

An HGF/SF antibody that may be used in this invention is AMG 102. “AMG 102” is an anti-HGF/SF antibody, as described in US Patent Publication No. 2005/0118643 and WO 2005/017107 which are herein incorporated by reference in its entirety, particularly in parts pertinent to AMG 102, its structure and properties, methods for making and using it, and other related antibodies. AMG 102 is identified in US 2005/0118643 and WO 2005/017107 as antibody 2.12.1.

Another HGF/SF antibody that may be used in this invention is L2G7. L2G7 is a humanized monoclonal anti-HGF/SF antibody, as described in WO 2005/107800, WO 2007/115049, and U.S. Pat. No. 7,494,650 and U.S. Pat. No. 7,220,410, which are herein incorporated by reference in its entirety, particularly in parts pertinent to AMG 102, its structure and properties, methods for making and using it, and other related antibodies.

A “pharmaceutically-acceptable derivative” denotes any salt, ester of a compound of this invention, or any other compound which upon administration to a patient is capable of providing (directly or indirectly) a compound of this invention, or a metabolite or residue thereof.

The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, example of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, ethanedisulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, β-hydroxybutyric, salicylic, galactaric and galacturonic acid. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, aistidine, glucamine, isopropylamine, lysine, morpholine, N-ethyl morpholine, piperazine, piperidine, triethylamine, trimethylamine All of these salts may be prepared by conventional means from the corresponding compound of the invention by reacting, for example, the appropriate acid or base with the compound of the invention. When a basic group and an acid group are present in the same molecule, a compound of the invention may also form internal salts.

Currently, standard treatment of primary tumors consists of surgical excision followed by either radiation or IV administered chemotherapy. The typical chemotherapy regime consists of either DNA alkylating agents, DNA intercalating agents, CDK inhibitors, or microtubule poisons. The chemotherapy doses used are just below the maximal tolerated dose and therefore dose limiting toxicities typically include, nausea, vomiting, diarrhea, hair loss, neutropenia and the like.

There are large numbers of antineoplastic agents available in commercial use, in clinical evaluation and in pre-clinical development, which would be selected for treatment of neoplasia by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents.

A first family of antineoplastic agents which may be used in combination with compounds of the present invention consists of antimetabolite-type/thymidilate synthase inhibitor antineoplastic agents. Suitable antimetabolite antineoplastic agents may be selected from but not limited to the group consisting of 5-FU, fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, Taiho UFT and uricytin.

A second family of antineoplastic agents which may be used in combination with compounds of the present invention consists of alkylating-type antineoplastic agents. Suitable alkylating-type antineoplastic agents may be selected from but not limited to the group consisting of Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)₂, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

A third family of antineoplastic agents which may be used in combination with compounds of the present invention consists of antibiotic-type antineoplastic agents. Suitable antibiotic-type antineoplastic agents may be selected from but not limited to the group consisting of Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-AL esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

A fourth family of antineoplastic agents which may be used in combination with compounds of the present invention consists of a miscellaneous family of antineoplastic agents, including tubulin interacting agents, topoisomerase II inhibitors, topoisomerase I inhibitors and hormonal agents, selected from but not limited to the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanlne derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides and Yamanouchi YM-534.

Alternatively, the present compounds may also be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RII retinamide, rituximab, romurtide, samarium (153 Sm) lexidronam, sargramostim, sizofuran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Alternatively, the present compounds may also be used with radiation. Alternatively, the present compounds may also be used in conjunction with agents used for hormonal therapy, such as for treatment of breast and prostate cancer. Examples include aromatase inhibitors (e.g. Arimidex (chemical name: anastrozole), Aromasin (chemical name: exemestane), and Femara (chemical name: letrozole)); Serms (selective estrogen-receptor modulators) such as tamoxifen; and ERDs (estrogen-receptor downregulators), e.g. Faslodex (chemical name: fulvestrant).

As will be appreciated, the dose of a combination of the present invention to be administered, the period of administration, and the general administration regime may differ between subjects depending on such variables as the severity of symptoms, the type of tumor to be treated, the mode of administration chosen, type of composition, size of a unit dosage, kind of excipients, the age and/or general health of a subject, and other factors well known to those of ordinary skill in the art.

Administration may include a single daily dose or administration of a number of discrete divided doses as may be appropriate. An administration regime may also include administration of one or more of the active agents, or compositions comprising same, as described herein. The period of administration may be variable.

It may occur for as long a period is desired.

Administration may include simultaneous administration of suitable agents or compositions or sequential administration of agents or compositions.

### Formulations

Also embraced within this invention is a class of pharmaceutical compositions comprising the active VEGFR inhibitors and/or the active c-Met inhibitors in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a tablet, capsule, suspension or liquid. It is contemplated that the pharmaceutical composition is made in the form of a dosage unit containing a particular amount of the active ingredient. Examples of such dosage units are tablets or capsules. For example, these may contain an amount of active ingredient from about 1 to 2000 mg, from about 1 to 800 mg. A suitable daily dose for a human or other mammal may vary widely depending on the condition of the patient and other factors, but, once again, can be determined using routine methods. For example, dosages from about 10 mg to about 150 mg, or about 25 to about 125 mg may be used. The therapeutically effective amount of VEGFR inhibitor in the composition can be chosen to be about 25 mg, about 50 mg, about 75 mg, about 100 mg, about 125 mg, or about 150 mg. The therapeutically effective amount of VEGFR inhibitor in the composition can be chosen to be about 50 mg dosed twice a day, or about 75 mg dosed twice a day, or about 100 mg dosed twice a day, or about 75 mg dosed once a day, or about 100 mg dosed once a day, or about 125 mg dosed once a day. The therapeutically effective amount of c-Met inhibitor in the composition can be chosen to be about 1 mg, about 2 mg, about 5 mg, about 10 mg, about 25 mg, about 50 mg, about 75 mg, about 100 mg, about 150 mg, about 250 mg, about 350 mg, or about 500 mg.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A daily dose of about 0.01 to 500 mg/kg, or between about 0.01 and about 50 mg/kg, or between about 0.01 and about 30 mg/kg body weight may be appropriate. The daily dose can be administered in one to four doses per day.

For therapeutic purposes, the active compounds of this invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

For pulmonary administration, the pharmaceutical composition may be administered in the form of an aerosol or with an inhaler including dry powder aerosol.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

While specific dosing for antibodies in accordance with the invention has not yet been determined, antibody can be administered with weekly doses in the range of about 0.5 mg/kg to about 30 mg/kg, from about 2 mg/kg to about 20 mg/kg. Antibody can be administered every two weeks with doses in the range of about 1 mg/kg to about 20 mg/kg, from about 3 mg/kg to about 20 mg/kg. The therapeutically effective amount of anti-HGF antibody in the composition can be chosen from about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg or about 20 mg.

Three distinct delivery approaches are expected to be useful for delivery of the antibodies in accordance with the invention. Conventional intravenous delivery, such as through a peripheral line or indwelling catheter over the length of time specified in the protocol, will presumably be the standard delivery technique for the majority of tumors. However, in connection with tumors in the peritoneal cavity, such as tumors of the ovaries, biliary duct, other ducts, and the like, intraperitoneal administration may prove favorable for obtaining high dose of antibody at the tumor and to minimize antibody clearance. In a similar manner certain solid tumors possess vasculature that is appropriate for regional perfusion. Regional perfusion will allow the obtention of a high dose of the antibody at the site of a tumor and will minimize short-term clearance of the antibody.

The antibody can be formulated in an aqueous buffer solution. The formulation may contain sodium chloride, sodium phosphate or sodium acetate at a physiological pH of about 5 to about 7.4. The formulation may or may not contain preservatives.

### Kits

The invention also provides kits comprising one or more HGF/SF:c-Met inhibitors and one or more VEGF inhibitors in accordance with the foregoing. The inhibitors may be disposed in the kits in one or more containers. Each such container may contain separately or in admixture one or more HGF/SF:c-Met inhibitors and one or more VEGF inhibitors in accordance with any of the foregoing. Typically, such kits are designed for medical use, and the inhibitors are comprised in pharmaceutically acceptable formulations. Among the contemplated kits are those comprising motesanib and AMG-102 and/or Amgen Compound 2 and/or Amgen Compound 3. Also among the contemplated embodiments are kits wherein the inhibitors are disposed in separate containers.

Further contemplated kits are those that comprise integrally thereto or as one or more separate documents, information pertaining to the contents or the kit and the use of the inhibitors. Also among further contemplated kits are those wherein the compositions are formulated for reconstitution in a diluent. In this regard, kits further comprising one or more containers of sterile diluent are contemplated Yet further contemplated embodiments include kits wherein at least one of the inhibitors is disposed in vials under partial vacuum sealed by a septum and suitable for reconstitution to form a formulation effective for parental administration.

Contemplated embodiments of the present invention also include kits that provide single-dose packaging of one or more of the inhibitors. Kits also include those that provide single and multi-chambered pre-filled syringes (e.g., liquid syringes and lyosyringes) for administering one or more of the inhibitors. Also contemplated are kits in which the syringes are preloaded.

The invention will now be further described with reference to the following non-limiting examples.

### Example 1

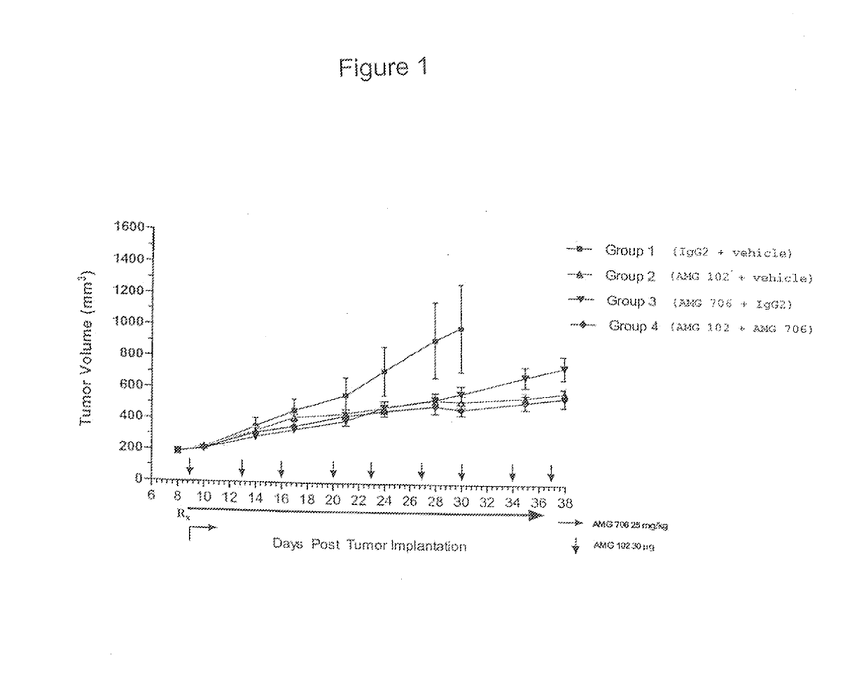

U118KR (human gliobastoma cells from the American Type Culture Collection (ATCC)) were expanded in culture, harvested and injected subcutaneously into 5-8 week old female athymic nude mice (Harlan Sprague Dawley, Inc) (n=10 per group). Treatment began on day nine (average tumor volume 180 mm³) with a VEGFR inhibitor, AMG 706, by oral gavage (25 mpk/dose/twice per day) or an HGF/SF:c-Met inhibitor, AMG 102 by intraperitoneal injection, (30 μg/dose/twice per week) or a combination of AMG 706 and AMG 102 at the same doses and schedules. Vehicle alone (acid water pH 2.0) and IgG2 isotype control antibody (30 μg/dose/twice per week) served as negative controls for the VEFGFR inhibitor and the HGF/SF:c-Met inhibitor, respectively. Progression of tumor growth was assessed by three dimensional caliper measurements and was recorded as a function of time. Statistical analysis was done by repeated measures analysis of variance (RMANOVA), followed by Scheffé post hoc testing for multiple comparisons. All treatments inhibited tumor growth when compared to the vehicle (p<0.02) at day 30. There was no enhanced efficacy when AMG 706 was combined with AMG 102 in this study. Body weights were not negatively impacted by any treatment. See FIG. 1.

### Example 2

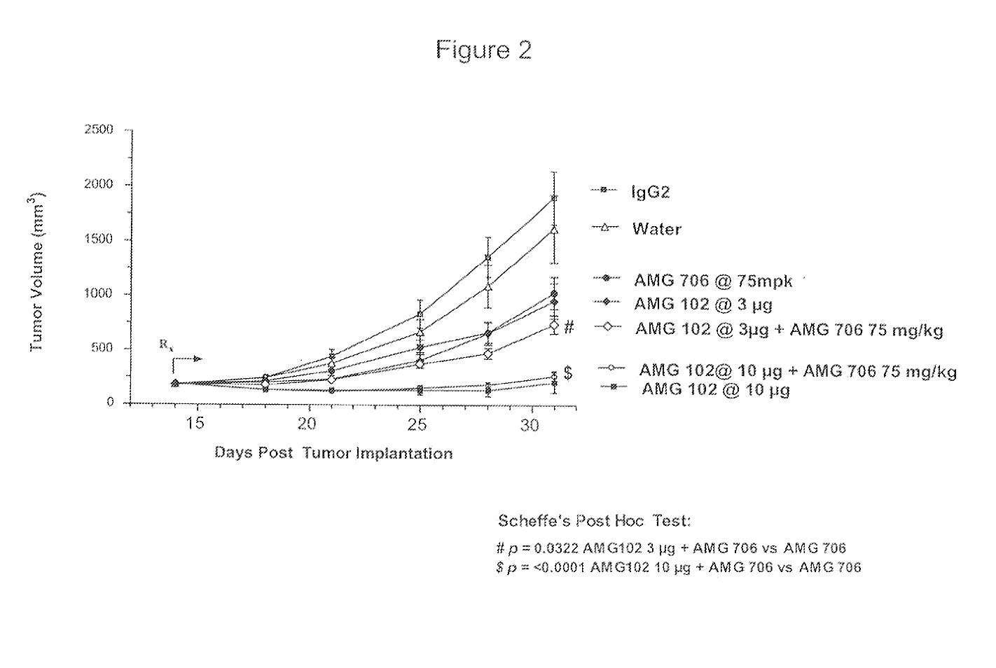

U-87 MG human glioblastoma tumor cells (from ATCC) were expanded in culture, harvested and injected subcutaneously into 5-8 week old female nude mice (CD1 NU/NU, Charles River Laboratories) (n=10/group). Treatment began on day 14 (average tumor volume 200 mm³) with a VEGFR inhibitor, AMG 706, by oral gavage (75 mg/kg/dose/once per day) or an HGF/SF:c-Met inhibitor, AMG 102 by intraperitoneal injection, (3 or 10 μg/dose/twice per week) or a combination of AMG 706 and AMG 102 at the same doses and schedules. Vehicle alone (acid water pH 2.0) and IgG2 isotype control antibody (30 μg/dose/twice per week) served as negative controls for the VEFGFR inhibitor and the HGF/SF:c-Met inhibitor, respectively. Progression of tumor growth was followed by three dimensional caliper measurements and recorded as a function of time. Initial statistical analysis was done by repeated measures analysis of variance (ANOVA), followed by Scheffé post hoc testing for multiple comparisons. Treatment with AMG 102 at 3 or 10 μg per dose significantly inhibited tumor growth compared to the isotype control group (p<0.0005). Treatment with AMG 706 at 75 mg/kg had no significant effect on tumor growth when compared with its vehicle control group. The combination of AMG 706 and AMG 102 at both doses had increased efficacy compared to the AMG 706 monotherapy group (p<0.05) but was not significant when compared to the AMG 102 monotherapy group. The combination of AMG 706 and AMG 102 provided no additional benefit compared to AMG 102 monotherapy. Body weights were not negatively impacted by any treatment. Arrow and Rx denote start of dosing. See FIG. 2.

### Example 3

MKN45 gastric carcinoma cells from the Japanese Health Science Research Resources Bank were expanded in culture, harvested and injected subcutaneously into 8 week old female CD1 nu/nu mice (Charles River Labs) (n=10/group). Administration of VEGFR inhibitor, Amgen Compound 1 (2-((1H-pyrrolo[2,3-b]pyridin-4-yl)methylamino)-5-fluoro-N-(2-methylbenzo[d]thiazol-5-yl)nicotinamide) (10 or 30 mpk/dose) or of c-Met inhibitor, Compound X (N-[4-(6,7-dimethoxyquinolin-4-yloxy)-3-fluorophenyl]-N-phenylactylthiourea) (10 mpk/dose) or by a combination of Amgen Compound 1 (10 or 30 mpk/dose) and Compound X (10 mpk/dose) began on day 18 post tumor cell implantation. Amgen Compound 1 was subsequently administered once daily by oral gavage (10 or 30 mpk) and Compound X was administered by oral gavage (10 mpk) once daily for the duration of the experiment. Progression of tumor growth was assessed by three dimensional caliper measurements and recorded as a function of time. Statistical analysis was performed by repeated measures analysis of variance (RMANOVA) followed by Scheffe post hoc testing for multiple comparisons. Vehicles (OraPlus, pH 2.0 and/or OraPlus, 1% Tween 80) were the negative controls for Amgen Compound 1 and Compound X, respectively. All treatment groups inhibited tumor growth compared to the vehicle (p<0.0222). Enhanced activity was observed when Amgen Compound 1 at 30 mpk was combined with Compound X at 10 mpk compared to either single agent alone (p≦0.0290). Enhanced activity was observed when Amgen Compound 1 at 10 mpk was combined with Compound X at 10 mpk, compared to Amgen Compound 1 (p<0.0001) but not to Compound X (p=0.0666). Body weights were not negatively impacted by any treatment. Arrow and Rx denote the start of dosing.

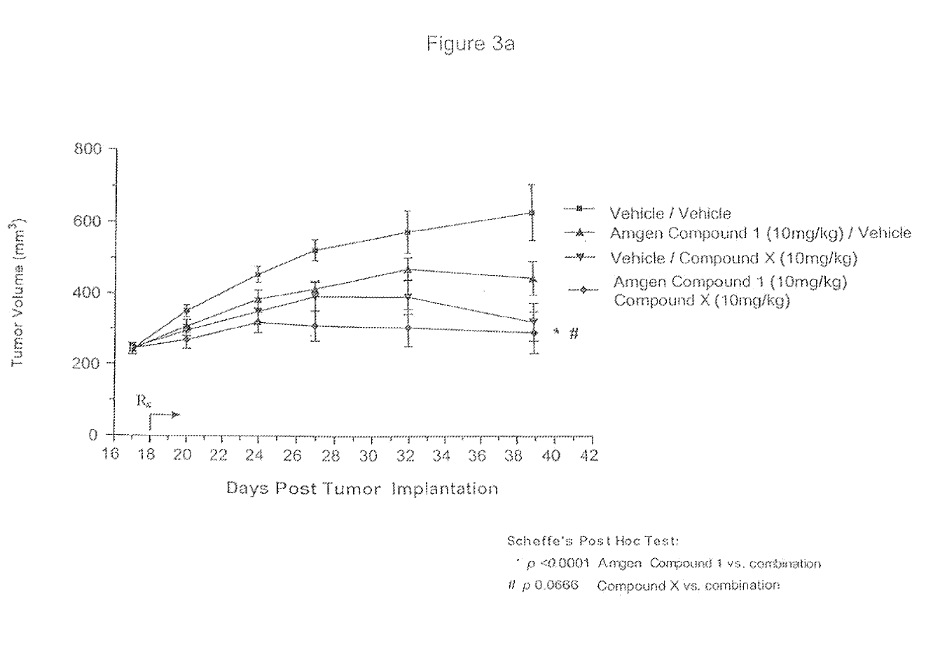

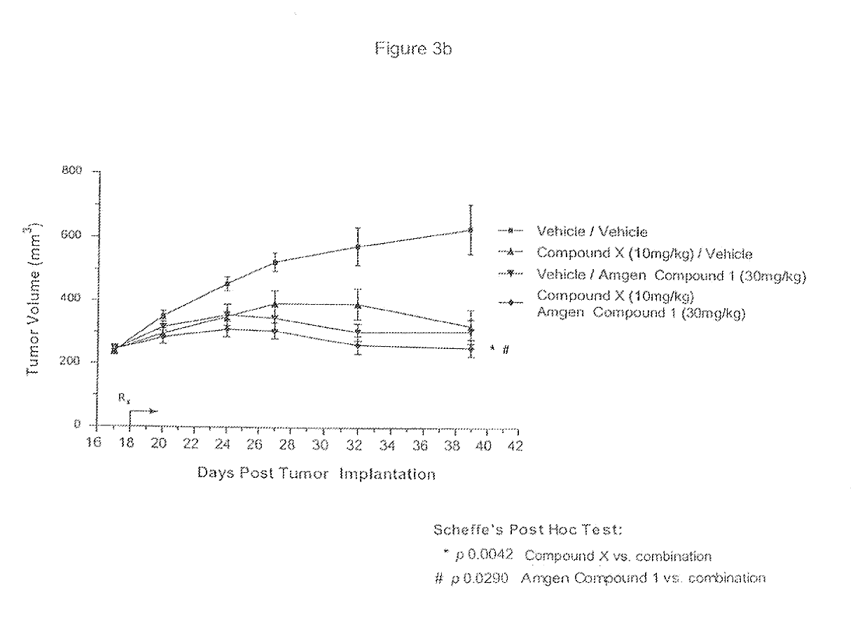

See FIG. 3A which shows the effect of Amgen Compound 1 in combination with 10 mpk/dose of Compound X. See FIG. 3B which shows the effect of Amgen Compound 1 in combination with 30 mpk/dose of Compound X.

### Example 4

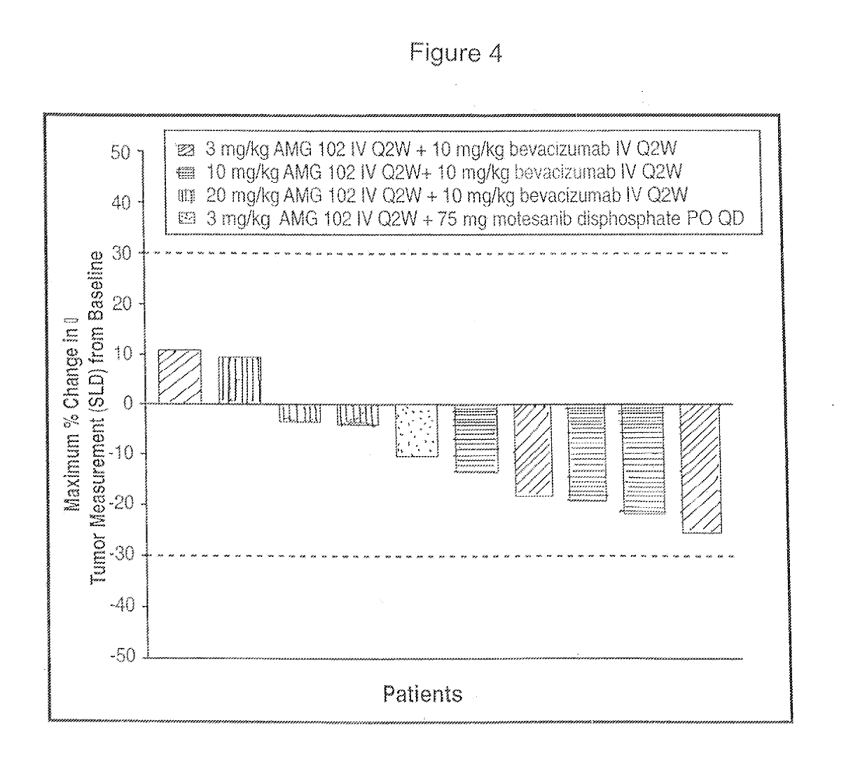

Fourteen patients (7 men, mean age 67) with advanced solid tumors enrolled in an open-label Phase 1b study. Prior treatment included radiotherapy (50%) and ≧3 lines of chemotherapy (71%). In this open-label Phase 1b study, 3 cohorts of 3-6 patients (patients) received an HGF/SF:c-Met inhibitor, AMG 102, an anti-HGF/SF antibody, (3, 10, or 20 mg/kg) in combination with a control VEGFR inhibitor, bevacizumab (AVASTIN®) (10 mg/kg), every 2 weeks (Q2W). Two patients received AMG 102 (3 mg/kg) IV Q2W+daily oral VEGFR inhibitor, motesanib (75 mg). Safety, tolerability, pharmacokinetics (PK), and tumor response were assessed in 10 patients. Four patients discontinued prior to post-dose tumor assessment (1 clinical progression, 2 Adverse Events, 1 Serious Adverse Event). See FIG. 4.

### Example 5

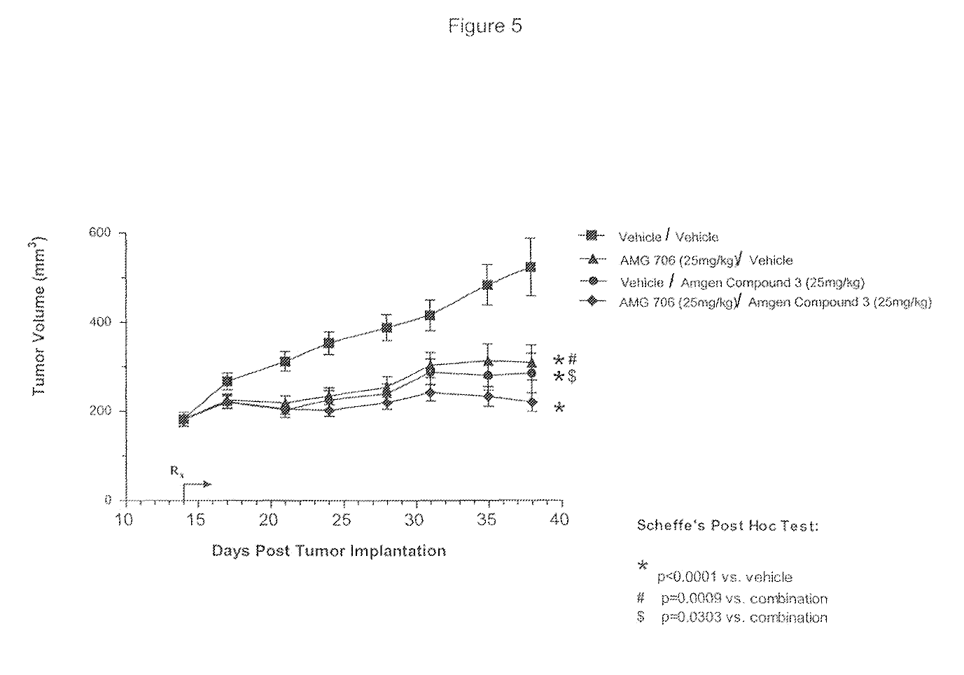

MKN45 (human gastric adenocarcinoma cancer cells from the Health Science Research Resources Bank, Japan) were expanded in culture, harvested and injected subcutaneously into six week old female athymic nude mice (Harlan Sprague Dawley, Inc) (n=10/group). Treatment began on day 14 (average tumor volume 180 mm³) with a VEGFR inhibitor, AMG 706, by oral gavage (25 mpk/dose/day) or a c-Met inhibitor, Amgen Compound 3, by oral gavage, (25 mpk/dose/once per day) or a combination of AMG 706 and Amgen Compound 3 at the same doses and schedules. Vehicles alone, acid water pH 2.0 and 2% HPMC 1% Tween 80 served as negative controls for the VEGFR inhibitor and the c-Met inhibitor, respectively. Tumor growth was assessed by three dimensional caliper measurements and was recorded as a function of time. Statistical analysis was performed by repeated measures analysis of variance (RMANOVA), followed by Scheffé post hoc testing for multiple comparisons. All treatments inhibited tumor growth when compared to the vehicle (p<0.05) from days 14-38. Enhanced activity was observed when AMG 706 was combined with Amgen Compound 3 compared to either single agent alone (p<0.0303). Body weights were not negatively impacted by any treatment. Arrow and Rx denote the start of dosing. See FIG. 5.

### Example 6

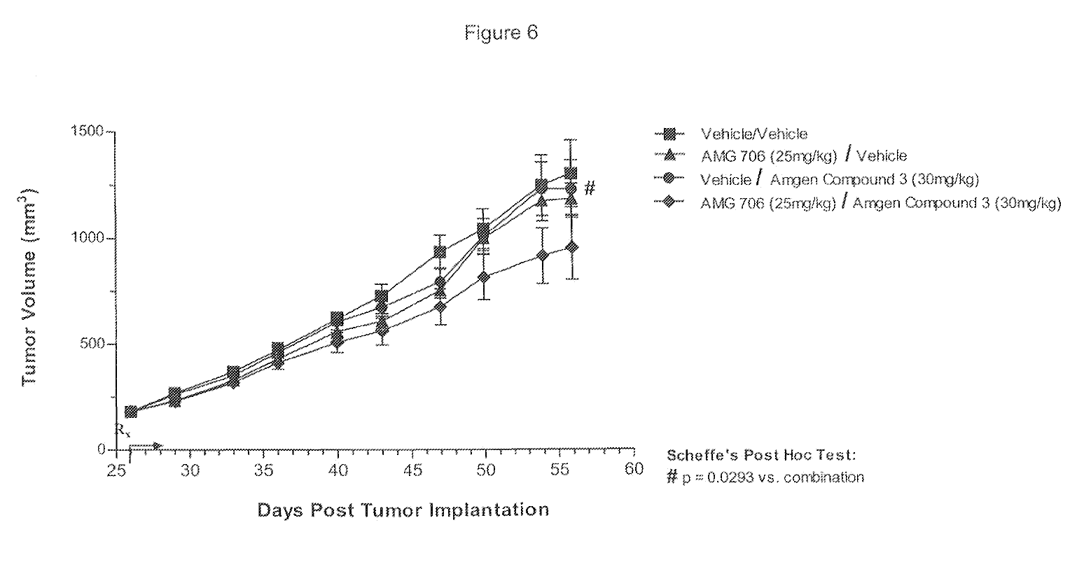

786-0-S4 (786-0 human renal cell adenocarcinoma cells from ATCC that were passaged in vivo) were expanded in culture, harvested and injected subcutaneously into 6 week old female athymic nude mice (Harlan Sprague Dawley) (n=10/group). Administration of the VEGFR inhibitor, AMG 706 (30 mpk/dose, PO), or the c-Met inhibitor, Amgen Compound 3 (25 mpk/dose, PO), or a combination of AMG 706 (30 mpk/dose, PO) and Amgen Compound 3 (25 mpk/dose, PO) began on day 25 post tumor cell implantation. AMG 706 was subsequently administered once daily by oral gavage (30 mpk/dose) and Amgen Compound 3 was administered once daily by oral gavage (25 mpk/dose) for the duration of the experiment. Progression of tumor growth was assessed by three dimensional caliper measurements and recorded as a function of time. Statistical analysis was performed by repeated measures analysis of variance (RMANOVA) followed by Scheffe post hoc testing for multiple comparisons. Vehicles (Acid water 2.2 and 2% HPMC 1% Tween 80) were the negative controls for AMG 706 and Amgen Compound 3, respectively. None of the treatment groups significantly inhibited tumor growth compared to the vehicle. Enhanced activity was observed when AMG 706 was combined with Amgen Compound 3 compared to AMG 706 as a single agent (p≦0.0293); however no enhanced activity was observed compared to Amgen Compound 3 as a single agent. Body weights were not negatively impacted by any treatment. Arrow and Rx denote the start of dosing. See FIG. 6.

### Example 7

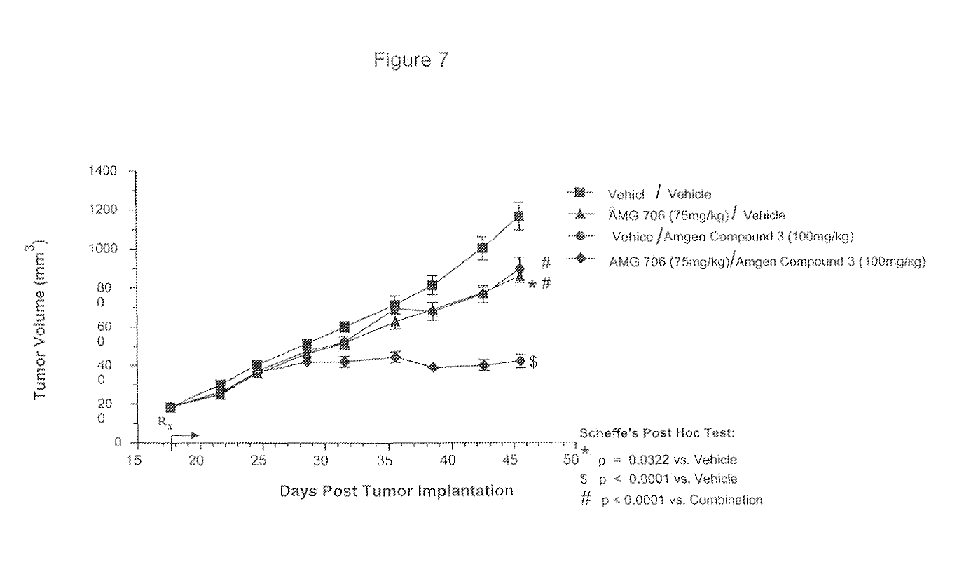

786-0-S4 (786-0 human renal cell adenocarcinoma cells from ATCC that were passaged in vivo) were expanded in culture, harvested and injected subcutaneously into 9 week old female athymic nude mice (Harlan Sprague Dawley) (n=10/group). Administration of the VEGFR inhibitor, AMG 706 (75 mpk/dose, PO), or the c-Met inhibitor, Amgen Compound 3 (100 mpk/dose, PO), or a combination of AMG 706 (75 mpk/dose, PO) and Amgen Compound 3 (100 mpk/dose, PO) began on day 20 post tumor cell implantation. AMG 706 was subsequently administered once daily by oral gavage (75 mpk/dose) and Amgen Compound 3 was administered once daily by oral gavage (100 mpk/dose) for the duration of the experiment. Progression of tumor growth was assessed by three dimensional caliper measurements and recorded as a function of time. Statistical analysis was performed by repeated measures analysis of variance (RMANOVA) followed by Scheffe post hoc testing for multiple comparisons. Vehicles (Acid water (pH 2.2) and 2% HPMC 1% Tween 80) served as negative controls for AMG 706 and Amgen Compound 3, respectively. The AMG 706 and combination treatment groups significantly inhibited tumor growth compared to the vehicle (p≦0.0322). The Amgen Compound 3 treatment group did not significantly inhibit tumor growth compared to the vehicle (p=0.0519). Enhanced activity was observed when AMG 706 at 75 mpk was combined with Amgen Compound 3 at 100 mpk compared to either single agent alone (p<0.0001). Body weights were not negatively impacted by any treatment. Arrow and Rx denote the start of dosing. See FIG. 7.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

All mentioned references, patents, applications and publications, are hereby incorporated by reference in their entirety, as if here written.

What is claimed is:

1. A method of treating cancer in a subject with an HGF/SF:c-Met inhibitor in combination with a VEGFR inhibitor selected from the group consisting of motesanib, bevacizumab, sorafenib, vatalanib, sunitinib, cediranib, and axitinib.

2. The method of claim 1 wherein the VEGFR inhibitor is motesanib.

3. The method of claim 1, wherein the VEGFR inhibitor is administered in a dose of about 25 mg to about 125 mg.

4. The method of claim 1, wherein the VEGFR inhibitor is administered in a dose of about 75 mg once a day.

5. The method of claim 1 wherein the VEGFR inhibitor is administered in a dose of about 125 mg once a day.

6. The method of claim 1, wherein the HGF/SF:c-Met inhibitor is an antibody.

7. The method of claim 7, wherein the HGF/SF:c-Met antibody is fully human.

8. The method of claim 8, wherein the HGF/SF:c-Met antibody is AMG-102.

9. The method of claim 7, wherein the HGF/SF:c-Met antibody is OA-5d5.

10. The method of claim 7, wherein the HGF/SF:c-Met antibody is humanized.

11. The method of claim 11, wherein the HGF/SF:c-Met antibody is L2G7.

12. The method of claim 7, wherein the anti-HGF/SF antibody is administered in a dose of about 2 mg/kg to about 30 mg/kg every two weeks.

13. The method of claim 1, wherein the HGF/SF:c-Met inhibitor is selected from the compounds of formula I
R—X—W—Y—R¹ I
enantiomers, diastereomers, salts solvates, and N-oxides thereof wherein
R is 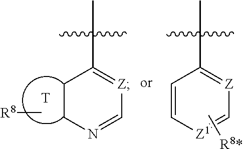
T is selected from phenyl, 5-6-membered heteroaryl, or 5-6 membered heterocyclyl;
Z is selected from N or CR⁷;
Z¹ is selected from N or CR⁷;
W is an substituted or unsubstituted phenyl, a substituted or unsubstituted benzomorpholinyl, a substituted or unsubstituted 6-membered nitrogen containing heteroaryl; a substituted or unsubstituted c₃₋₇cycloalkyl, c₁₋₆alkyl and c₁₋₆alkynyl;
X is selected from O, S, S(═O), SO₂, NR² and CR³R⁴;
Y is selected from —NR^{a}C(═O)—(CR³R⁴)ₚ—, —NR^{a}C(═S)—(CR³R⁴)ₚ—, —NR^{a}—(CR³R⁴)ₚ—, —NR^{a}—(CR³R⁴)ₚC(═O)—, —NR^{a}—(CR³R⁴)ₚC(═S)—, —NR^{a}S(═O)ₜ—, —NR^{a}S(═O)ₜ—(CR³R⁴)ₚ—, —C(═O)NR^{a}—(CR³R⁴)ₚ—, and —NR^{a}—(CR³R⁴)ₚ—S(═O)ₜ—, and where W is benzomorpholinyl Y may further include —C(═O);
R^{a} is selected from H, alkyl, heterocyclyl, aryl, arylalkyl, heterocyclylalkyl, cycloalkyl, cycloalkylalkyl, alkenyl and alkynyl; wherein R^{a} is optionally substituted;
R¹ is a partially unsaturated or saturated ring selected from 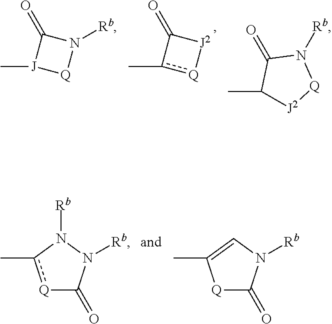
wherein J is N or CR^{4a};
J² is O or CR^{4a}R^{4a};
Q is a 1-5 membered saturated or partially unsaturated alkyl chain, or a 2-5 membered saturated or partially unsaturated heteroalkyl chain;
R¹ is optionally fused with an optionally substituted phenyl or an optionally substituted 5-6 membered heterocyclyl ring;
wherein R¹ is optionally substituted with one or more substituents independently selected from H, halo, hydroxyl, R^{5a}R^{a}N—, R^{5a}R^{a}N—C₁₋₆ alkyl, R⁵(S═O)—C₁₋₆ alkyl, NR⁵R^{5a}—(C═O)—C₁₋₆ alkyl, optionally substituted alkyl, alkenyl hydroxyalkyl, C₁₋₆ alkoxy-C₁₋₆ alkyl, alkenylalkyl, C₁₋₆ alkylthio-C₁₋₃ alkyl, —C₁₋₆ alkyl-NR^{a}—C(═O)—OR⁵, —C₁₋₃ alkyl-NR^{a}—(C═O)—R⁵, —C₁₋₃ alkyl-C(═O)—C₁₋₃ alkyl, aminoalkyl, hydroxy-substituted aminoalkyl, hydroxy-substituted haloalkyl, (heterocyclo)hydroxyalkyl, haloC₁₋₆-alkyl, azidoalkyl, optionally substituted aryl-C₁₋₆ alkyl, optionally substituted 5-6-membered heterocyclyl-C₁₋₆ alkyl, optionally substituted C₁₋₆-alkyl, optionally substituted C₃₋₇ cycloalkyl, optionally substituted 5-6 membered heterocyclyl, optionally substituted 5-10 membered heteroaryl, optionally, optionally substituted C₃₋₆ cycloalkyl, substituted heteroarylalkyl, optionally substituted arylalkyl, and optionally substituted C₆₋₁₀ aryl;
R^{b} is independently selected at each occurrence from H, optionally substituted arylalkyl, optionally substituted 5-6-membered heterocyclyl-C₁₋₃ alkyl, optionally substituted C₁₋₆-alkyl, optionally substituted 5-6 membered heterocyclyl, optionally substituted C₆₋₁₀ aryl, optionally substituted C₆₋₁₀ heteroaryl, optionally substituted C₃₋₆ cycloalkyl, and R^{a}R^{5a}N—C₁₋₃alkyl;
R² is selected from H, alkyl, haloalkyl, aryl, heterocyclyl, arylalkyl, heterocyclylalkyl, cycloalkyl, cycloalkylalkyl, alkenyl, alkynyl and R⁵-carbonyl;
R³ and R⁴ are each independently selected from H, alkyl, aryl, heterocyclyl, arylalkyl, heterocyclylalkyl, haloalkyl, cycloalkyl, cycloalkylalkyl, R⁶ and alkyl substituted with R⁶; alternatively R³ and R⁴, together with the carbon atom they are attached to, form an optionally substituted 3-6 membered ring;
R^{3a} is absent or is selected from H, alkyl, aryl, heterocyclyl, arylalkyl, heterocyclylalkyl, cycloalkyl, cycloalkylalkyl, R⁶ and alkyl substituted with R⁶;
R^{4a} is absent or is selected from H, halo, —OR⁵—NR^{a}R⁵, alkyl, aryl, heterocyclyl, arylalkyl, heterocyclylalkyl, cycloalkyl, cycloalkylalkyl, R⁶ and alkyl substituted with R⁶;
R⁵ is independently selected at each occurrence from H, alkyl, haloalkyl, hydroxyalkyl, alkoxyalkyl, alkylaminoalkyl, alkylthioalkyl, arylalkyl, heterocyclylalkyl, cycloalkylalkyl, aryl, heterocyclyl, alkenyl, alkynyl and cycloalkyl;
R^{5a} is independently selected at each occurrence from H, alkyl, haloalkyl, arylalkyl aminoalkyl, heterocyclylalkyl, cycloalkylalkyl, aryl, heterocyclyl, alkenyl, alkynyl and cycloalkyl;
or when R⁵ and R^{a}, or R^{5a} and R^{a} are bonded to the same nitrogen atom, R^{a} and R⁵, or R^{a} and R^{5a} may independently optionally combine to form a heterocyclo ring.
R⁶ is selected from cyano, —OR², —SR², halo, —SO₂R², —C(═O)R², —SO₂NR²R⁵, —NR⁵C(═O)OR², —NR⁵C(═O)NR⁵R², —NR⁵C(═O)R², —CO₂R², —C(═O)NR²R⁵ and —NR²R⁵;
R⁷ is selected from H, halo, cyano, —C(═O)NR^{a}R⁵ and alkyl;
R⁸ is one or more substituents independently selected at each occurrence from H, cyano, hydroxyl, halo, optionally substituted heterocyclyl, —C(═O)NR^{a}R⁵, —OC(═O)NR^{a}R⁵, —NR^{a}C(═O)OR⁵, —NR^{a}C(═O)—R⁵, R⁵R^{a}N—O₂S—, R⁵O₂S—, R⁵O₂SR^{a}N—, R⁵R^{a}N—, alkyl, aminoalkyl, alkylaminoalkyl, alkoxyalkyl, phenylalkyl, heterocyclylalkyl, alkoxy, haloalkoxy, alkylaminoalkoxy, arylalkoxy, heterocyclylalkoxy, cycloalkylalkoxy, heterocyclyl(hydroxyalkoxy), cycloalkyl(hydroxyalkoxy), aryl(hydroxyalkoxy), alkoxyalkoxy, aryloxyalkoxy, heterocyclyloxyalkoxy, cycloalkyloxyalkoxy, aryloxy, heterocyclyloxy, cycloalkyloxy; aryl and heteroaryl, alternatively where R⁸ comprises an NR^{a}R⁵ moiety R^{a} and R⁵, together with the nitrogen atom they are attached to, may optionally form a substituted or unsubstituted 4-6 membered ring;
R⁸* is one or more substituents independently selected at each occurrence from H, cyano, hydroxyl, halo, optionally substituted heterocyclyl, —NR^{a}C(═O)NR^{a}R⁵, NR^{a}C(═NR^{b})—NR^{a}R⁵, NR^{a}C(═S)NR^{a}R⁵, —OC(═O)NR^{a}R⁵, —NR^{a}C(═O)OR⁵, —NR^{a}C(═O)—R⁵, R⁵R^{a}N—O₂S—, R⁵O₂S—, R⁵O₂SR^{a}N—, R⁵R^{a}N—, alkyl, aminoalkyl, alkylaminoalkyl, alkoxyalkyl, phenylalkyl, heterocyclylalkyl, alkoxy, haloalkoxy, alkylaminoalkoxy, arylalkoxy, heterocyclylalkoxy, cycloalkylalkoxy, heterocyclyl(hydroxyalkoxy), cycloalkyl(hydroxyalkoxy), aryl(hydroxyalkoxy), alkoxyalkoxy, aryloxyalkoxy, heterocyclyloxyalkoxy, cycloalkyloxyalkoxy, aryloxy, heterocyclyloxy, and cycloalkyloxy;
alternatively where R^{8a}R⁸* comprises an NR^{a}R⁵ moiety R^{a} and R⁵, together with the nitrogen atom they are attached to, may optionally form a substituted or unsubstituted 4-6 membered ring;
p is 0, 1, 2, or 3; and
t is 0, 1 or 2;
wherein each alkyl, aryl, heteroaryl, cycloalkyl, alkenyl, alkynyl, heterocyclyl, and alkoxy moiety of any R, R¹, R², R³, R⁴, R⁵, R⁷, R⁸, R⁸*, and R^{a} is optionally independently substituted with one or more groups independently selected at each occurrence from halo, oxo, —NR^{a}R⁵, —OR^{5a}, —CO₂R⁵, —C(═O)R⁵, (C₁-C₆)alkylamino, —NH—N═NH, (C₁-C₆)alkyl, (C₁-C₆)alkynyl, (C₃-C₆)cycloalkyl, (C₁-C₆)haloalkyl, di(C₁-C₆)alkylamino, (C₁-C₆)alkylamino-(C₁-C₆)alkyl, (C₁-C₆)hydroxyalkylamino, (C₁-C₆)alkylamino-(C₁-C₆)alkylamino, phenyl, heterocyclic, heteroaryl, —(CR³R⁴)ₚ alkyl-S(═O)-alkyl, and —(CR³R⁴)ₚalkyl-S(O)₂-alkyl.

14. The method of claim 13, wherein the HGF/SF:c-Met inhibitor is selected from the group consisting of:
N-(4-(6,7-dimethoxyquinolin-4-yloxy)-3-fluorophenyl)-1-methyl-3-oxo-2-phenyl-5-(pyridin-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyrrolidin-1-ylmethyl)-2,3-dihydro-1H-pyrazole-4-carboxamide;
N-(4-(6,7-dimethoxyquinolin-4-yloxy)-3-fluorophenyl)-5-((ethyl(methyl)amino)methyl)-1-methyl-3-oxo-2-phenyl-2,3-dihydro-1H-pyrazole-4-carboxamide;
N-(4-(6,7-dimethoxyquinolin-4-yloxy)-3-fluorophenyl)-5-((dimethylamino)methyl)-1-methyl-3-oxo-2-phenyl-2,3-dihydro-1H-pyrazole-4-carboxamide;
5-(aminomethyl)-N-(3-fluoro-4-(7-methoxyquinolin-4-yloxy)phenyl)-1-methyl-3-oxo-2-phenyl-2,3-dihydro-1H-pyrazole-4-carboxamide;
tert-butyl (4-((3-fluoro-4-(7-methoxyquinolin-4-yloxy)phenyl)carbamoyl)-1-methyl-3-oxo-2-phenyl-2,3-dihydro-1H-pyrazol-5-yl)methylcarbamate;
N-(5-(7-methoxyquinolin-4-yloxy)pyridin-2-yl)-1-methyl-3-oxo-2-phenyl-5-(pyrrolidin-1-ylmethyl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyrrolidin-1-ylmethyl)-2,3-dihydro-1H-pyrazole-4-carboxamide;
N-(3-fluoro-4-(7-methoxyquinolin-4-yloxy)phenyl)-5-methyl-3-oxo-2-phenyl-1-((tetrahydrofuran-2-yl)methyl)-2,3-dihydro-1H-pyrazole-4-carboxamide;
5-((ethyl(methyl)amino)methyl)-N-(3-fluoro-4-(7-methoxyquinolin-4-yloxy)phenyl)-1-methyl-3-oxo-2-phenyl-2,3-dihydro-1H-pyrazole-4-carboxamide;
2-benzyl-N-(5-(7-methoxyquinolin-4-yloxy)pyridin-2-yl)-1-methyl-3-oxo-5-(pyridin-4-yl)-2,3-dihydro-1H-pyrazole-4-carboxamide;
2-benzyl-N-(3-fluoro-4-(7-methoxyquinolin-4-yloxy)phenyl)-1-methyl-3-oxo-5-(pyridin-4-yl)-2,3-dihydro-1H-pyrazole-4-carboxamide;
(S)-N-(5-(7-methoxyquinolin-4-yloxy)pyridin-2-yl)-1-methyl-3-oxo-2-(1-phenylethyl)-5-(pyridin-4-yl)-2,3-dihydro-1H-pyrazole-4-carboxamide;
(S)-N-(3-fluoro-4-(7-methoxyquinolin-4-yloxy)phenyl)-1-methyl-3-oxo-2-(1-phenylethyl)-5-(pyridin-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyridin-4-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyridin-4-yl)-2,3-dihydro-1H-pyrazole-4-carboxamide;
N-(5-(6,7-dimethoxyquinolin-4-yloxy)pyridin-2-yl)-1-methyl-3-oxo-2-phenyl-5-(pyridin-4-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyridin-2-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyridin-2-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(tetrahydro-2H-pyran-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(tetrahydro-2H-pyran-4-yl)-2,3-dihydro-1H-pyrazole-4-carboxamide;
1-methyl-N-(5-((7-(methyloxy)-4-quinolinyl)oxy)-2-pyridinyl)-5-(2-methyl-1,3-thiazol-4-yl)-3-oxo-2-phenyl-2,3-dihydro-1H-pyrazole-4-carboxamide;
N-(5-((6,7-bis(methyloxy)-4-quinolinyl)oxy)-2-pyridinyl)-1-methyl-5-(5-methyl-3-isoxazolyl)-3-oxo-2-phenyl-2,3-dihydro-1H-pyrazole-4-carboxamide;
1-methyl-5-(5-methyl-3-isoxazolyl)-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-1-methyl-5-(5-methyl-3-isoxazolyl)-3-oxo-2-phenyl-2,3-dihydro-1H-pyrazole-4-carboxamide;
1-methyl-N-(5-((7-(methyloxy)-4-quinolinyl)oxy)-2-pyridinyl)-3-oxo-2-phenyl-5-(2-pyrazinyl)-2,3-dihydro-1H-pyrazole-4-carboxamide;
N-(3-fluoro-4-((7-(methyloxy)-4-quinolinyl)oxy)phenyl)-1-methyl-3-oxo-2-phenyl-5-(2-pyrazinyl)-2,3-dihydro-1H-pyrazole-4-carboxamide;
N-(5-((6,7-bis(methyloxy)-4-quinolinyl)oxy)-2-pyridinyl)-1-methyl-3-oxo-2-phenyl-5-(2-pyrazinyl)-2,3-dihydro-1H-pyrazole-4-carboxamide;
N-(5-((6,7-bis(methyloxy)-4-quinolinyl)oxy)-2-pyridinyl)-1-methyl-5-(2-methyl-1,3-thiazol-4-yl)-3-oxo-2-phenyl-2,3-dihydro-1H-pyrazole-4-carboxamide;
N-(3-fluoro-4-((7-(methyloxy)-4-quinolinyl)oxy)phenyl)-1-methyl-5-(2-methyl-1,3-thiazol-4-yl)-3-oxo-2-phenyl-2,3-dihydro-1H-pyrazole-4-carboxamide;
N-(4-(6,7-dimethoxyquinolin-4-yloxy)-3-fluorophenyl)-N,1,5-trimethyl-3-oxo-2-phenyl-2,3-dihydro-1H-pyrazole-4-carboxamide;
2-(3-chlorophenyl)-N-(4-(6,7-dimethoxyquinolin-4-yloxy)-3-fluorophenyl)-1,5-dimethyl-3-oxo-2,3-dihydro-1H-pyrazole-4-carboxamide;
2-(3-chlorophenyl)-N-(5-(6,7-dimethoxyquinolin-4-yloxy)pyridin-2-yl)-1,5-dimethyl-3-oxo-2,3-dihydro-1H-pyrazole-4-carboxamide;
N-(4-(6,7-dimethoxyquinolin-4-yloxy)-3-fluorophenyl)-1,5-dimethyl-3-oxo-2-p-tolyl-2,3-dihydro-1H-pyrazole-4-carboxamide;
N-(4-(6,7-dimethoxyquinolin-4-yloxy)-3-fluorophenyl)-2-(4-fluorophenyl)-1,5-dimethyl-3-oxo-2,3-dihydro-1H-pyrazole-4-carboxamide;
N-(5-(6,7-dimethoxyquinolin-4-yloxy)pyridine-2-yl)-1,5-dimethyl-3-oxo-2-p-tolyl-2,3-dihydro-1H-pyrazole-4-carboxamide;
N-(5-(6,7-dimethoxyquinolin-4-yloxy)pyridin-2-yl)-2-(4-fluorophenyl)-1,5-dimethyl-3-oxo-2,3-dihydro-1H-pyrazole-4-carboxamide;
2-(3-chlorophenyl)-N-(5-(7-methoxyquinolin-4-yloxy)pyridin-2-yl)-1,5-dimethyl-3-oxo-2,3-dihydro-1H-pyrazole-4-carboxamide;
N-(5-(7-methoxyquinolin-4-yloxy)pyridin-2-yl)-1,5-dimethyl-3-oxo-2-p-tolyl-2,3-dihydro-1H-pyrazole-4-carboxamide;
2-(2-chlorophenyl)-N-(5-(6,7-dimethoxyquinolin-4-yloxy)pyridin-2-yl)-1,5-dimethyl-3-oxo-2,3-dihydro-1H-pyrazole-4-carboxamide;
2-(2-chlorophenyl)-N-(4-(6,7-dimethoxyquinolin-4-yloxy)-3-fluorophenyl)-1,5-dimethyl-3-oxo-2,3-dihydro-1H-pyrazole-4-carboxamide;
2-(2-chlorophenyl)-N-(3-fluoro-4-(7-methoxyquinolin-4-yloxy)phenyl)-1,5-dimethyl-3-oxo-2,3-dihydro-1H-pyrazole-4-carboxamide;
N-(3-fluoro-4-(7-methoxyquinolin-4-yloxy)phenyl)-2-(4-fluorophenyl)-1,5-dimethyl-3-oxo-2,3-dihydro-1H-pyrazole-4-carboxamide;
2-(3-chlorophenyl)-N-(3-fluoro-4-(7-methoxyquinolin-4-yloxy)phenyl)-1,5-dimethyl-3-oxo-2,3-dihydro-1H-pyrazole-4-carboxamide;
N-(6-(6,7-dimethoxyquinolin-4-yloxy)pyridin-3-yl)-1,5-dimethyl-3-oxo-2-phenyl-2,3-dihydro-1H-pyrazole-4-carboxamide;
N-(2-chloro-4-(6,7-dimethoxyquinolin-4-yloxy)phenyl)-1,5-dimethyl-3-oxo-2-phenyl-2,3-dihydro-1H-pyrazole-4-carboxamide;
2-benzyl-N-(4-(6,7-dimethoxyquinolin-4-yloxy)-3-fluorophenyl)-1,5-dimethyl-3-oxo-2,3-dihydro-1H-pyrazole-4-carboxamide;
2-benzyl-N-(5-(6,7-dimethoxyquinolin-4-yloxy)pyridin-2-yl)-1,5-dimethyl-3-oxo-2,3-dihydro-1H-pyrazole-4-carboxamide;
N-(5-(7-methoxyquinolin-4-yloxy)pyridin-2-yl)-1-methyl-3-oxo-2-phenyl-2,3-dihydro-1H-pyrazole-4-carboxamide;
N-(4-(6,7-dimethoxyquinolin-4-yloxy)-3-fluorophenyl)-1-(2-hydroxy-2-methylpropyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-3-oxo-1-(2-oxobutyl)-2-phenyl-2,3-dihydro-1H-pyrazole-4-carboxamide;
N-(3-fluoro-4-(7-methoxyquinolin-4-yloxy)phenyl)-5-methyl-1-(3-methyl-2-oxobutyl)-3-oxo-2-phenyl-2,3-dihydro-1H-pyrazole-4-carboxamide;
(R)-N-(3-fluoro-4-(7-methoxyquinolin-4-yloxy)phenyl)-1-(2-hydroxybutyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2R,3R)-3-hydroxybutan-2-yl)-5-methyl-3-oxo-2-phenyl-2,3-dihydro-1H-pyrazole-4-carboxamide;
1-((2R,3R)-3-hydroxybutan-2-yl)-N-(5-(7-methoxyquinolin-4-yloxy)pyridin-2-yl)-5-methyl-3-oxo-2-phenyl-2,3-dihydro-1H-pyrazole-4-carboxamide;
(S)-1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
(R)-1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
(S)-N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
(R)-N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-1-((3-methyl-2-oxooxazolidin-5-yl)methyl)-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-(methylamino)propyl)-5-methyl-3-oxo-2-phenyl-2,3-dihydro-1H-pyrazole-4-carboxamide;
1-(3-chloro-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-2-methylbutyl)-5-methyl-3-oxo-2-phenyl-2,3-dihydro-1H-pyrazole-4-carboxamide;
1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-morpholinopropyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-1-(oxazolidin-5-ylmethyl)-3-oxo-2-phenyl-2,3-dihydro-1H-pyrazole-4-carboxamide;
(S)-N-(3-fluoro-4-(7-methoxyquinolin-4-yloxy)phenyl)-1-(2-hydroxybutyl)-5-methyl-3-oxo-2-phenyl-2,3-dihydro-1H-pyrazole-4-carboxamide;
1-(3-amino-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-2-methylpropyl)-5-methyl-3-oxo-2-phenyl-2,3-dihydro-1H-pyrazole-4-carboxamide;
(R)-1-(2-hydroxypropyl)-N-(5-(7-methoxyquinolin-4-yloxy)pyridin-2-yl)-5-methyl-3-oxo-2-phenyl-2,3-dihydro-1H-pyrazole-4-carboxamide;
1-(3-(dimethylamino)-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
(R)-N-(3-fluoro-4-(7-methoxyquinolin-4-yloxy)phenyl)-1-(2-hydroxypropyl)-5-methyl-3-oxo-2-phenyl-2,3-dihydro-1H-pyrazole-4-carboxamide;
(R)-N-(4-(6,7-dimethoxyquinolin-4-yloxy)-3-fluorophenyl)-1-(2-hydroxypropyl)-5-methyl-3-oxo-2-phenyl-2,3-dihydro-1H-pyrazole-4-carboxamide;
1-(2-hydroxypropyl)-N-(5-(7-methoxyquinolin-4-yloxy)pyridin-2-yl)-5-methyl-3-oxo-2-phenyl-2,3-dihydro-1H-pyrazole-4-carboxamide;
N-(5-(6,7-dimethoxyquinolin-4-yloxy)pyridin-2-yl)-1-(2-hydroxy-2-methylpropyl)-5-methyl-3-oxo-2-phenyl-2,3-dihydro-1H-pyrazole-4-carboxamide;
(R)-2-(3-chlorophenyl)-N-(3-fluoro-4-(7-methoxyquinolin-4-yloxy)phenyl)-1-(2-hydroxypropyl)-5-methyl-3-oxo-2,3-dihydro-1H-pyrazole-4-carboxamide;
(R)-2-(3-chlorophenyl)-1-(2-hydroxypropyl)-N-(5-(7-methoxyquinolin-4-yloxy)pyridin-2-yl)-5-methyl-3-oxo-2,3-dihydro-1H-pyrazole-4-carboxamide;
(R)-N-(3-fluoro-4-(7-methoxyquinolin-4-yloxy)phenyl)-2-(4-fluorophenyl)-1-(2-hydroxypropyl)-5-methyl-3-oxo-2,3-dihydro-1H-pyrazole-4-carboxamide 1-(2-hydroxy-2-methylpropyl)-N-(5-(1-oxo-7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-hydroxyquinolin-4-yloxy)phenyl)-1-(2-hydroxy-2-methylpropyl)-5-methyl-3-oxo-2-phenyl-2,3-dihydro-1H-pyrazole-4-carboxamide;
1-(2-hydroxy-2-methylpropyl)-N-(5-(7-hydroxyquinolin-4-yloxy)pyridin-2-yl)-5-methyl-3-oxo-2-phenyl-2,3-dihydro-1H-pyrazole-4-carboxamide;
N-(4-(6-ethyl-7-methoxyquinolin-4-yloxy)-3-fluorophenyl)-1,5-dimethyl-3-oxo-2-phenyl-2,3-dihydro-1H-pyrazole-4-carboxamide;
N-(3-fluoro-4-(7-methoxyquinolin-4-yloxy)phenyl)-1,5-dimethyl-3-oxo-2-phenyl-2,3-dihydro-1H-pyrazole-4-carboxamide;
N-(3-fluoro-4-(7-Methoxyquinolin-4-yloxy)phenyl)-1,2-dimethyl-3-oxo-5-phenyl-2,3-dihydro-1H-pyrazole-4-carboxamide;
N-(5-(7-methoxyquinolin-4-yloxy)pyridin-2-yl)-1,2-dimethyl-3-oxo-5-phenyl-2,3-dihydro-1H-pyrazole-4-carboxamide;
N-(4-(6,7-dimethoxyquinolin-4-yloxy)-3-fluorophenyl)-1,2-dimethyl-3-oxo-5-phenyl-2,3-dihydro-1H-pyrazole-4-carboxamide;
N-(5-(7-methoxyquinolin-4-yloxy)pyridin-2-yl)-1,5-dimethyl-3-oxo-2-phenyl-2,3-dihydro-1H-pyrazole-4-carboxamide;
(R)-1-(2-hydroxypropyl)-N-(5-(7-methoxyquinolin-4-yloxy)pyridin-2-yl)-2-methyl-3-oxo-5-phenyl-2,3-dihydro-1H-pyrazole-4-carboxamide;
(R)-N-(3-fluoro-4-(7-methoxyquinolin-4-yloxy)phenyl)-1-(2-hydroxypropyl)-2-methyl-3-oxo-5-phenyl-2,3-dihydro-1H-pyrazole-4-carboxamide
(S)-N-(3-fluoro-4-(6-methoxyquinolin-4-yloxy)phenyl)-1-(2-hydroxypropyl)-5-methyl-3-oxo-2-phenyl-2,3-dihydro-1H-pyrazole-4-carboxamide;
1-(2-aminoethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide 1-(2-(1,3-dioxo-1,3-dihydro-2H-isoindol-2-yl)ethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-(2-aminoethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
5-methyl-N-(5-((7-(methyloxy)-4-quinolinyl)oxy)-2-pyridinyl)-3-oxo-2-phenyl-1-(phenylmethyl)-2,3-dihydro-1H-pyrazole-4-carboxamide 1-benzyl-N-(3-fluoro-4-(7-methoxyquinolin-4-yloxy)phenyl)-5-methyl-3-oxo-2-phenyl-2,3-dihydro-1H-pyrazole-4-carboxamide;
5-methyl-1-(2-(methyloxy)ethyl)-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-5-methyl-1-(2-(methyloxy)ethyl)-3-oxo-2-phenyl-2,3-dihydro-1H-pyrazole-4-carboxamide;
1-(2-hydroxyethyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
1-(2R)-2-fluoropropyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
(S)-1-(2-(dimethylamino)propyl)-N-(3-fluoro-4-(7-methoxyquinolin-4-yloxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-3-oxo-2-phenyl-1-(2-(1-pyrrolidinyl)ethyl)-2,3-dihydro-1H-pyrazole-4-carboxamide;
1-((2S)-2-fluoropropyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-1-((2S)-2-fluoropropyl)-5-methyl-3-oxo-2-phenyl-2,3-dihydro-1H-pyrazole-4-carboxamide;
1-((2S)-2-(acetylamino)propyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-((2S)-2-aminopropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-((2S)-2-azidopropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-1-(2-hydroxyethyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-3-oxo-2-phenyl-1-propyl-2,3-dihydro-1H-pyrazole-4-carboxamide;
N-(4-((6,7-bis(methyloxy)-4-quinolinyl)oxy)-3-fluorophenyl)-1-(2R)-2-hydroxypropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-1-((2S)-2-hydroxypropyl)-5-methyl-3-oxo-2-phenyl-2,3-dihydro-1H-pyrazole-4-carboxamide;
5-methyl-N-(5-(97-(methyloxy)-4-quinolinyl)oxy)-2-pyridinyl)-1-(2-methylpropyl)-3-oxo-2-phenyl-2,3-dihydro-1H-pyrazole-4-carboxamide;
5-methyl-N-(5-((7-(methyloxy)-4-quinolinyl)oxy)-2-pyridinyl)-3-oxo-2-phenyl-1-propyl-2,3-dihydro-1H-pyrazole-4-carboxamide;
N-(3-fluoro-4-((7-(methyloxy)-4-quinolinyl)oxy)phenyl)-5-methyl-3-oxo-1-(2-oxopropyl)-2-phenyl-2,3-dihydro-1H-pyrazole-4-carboxamide;
1-(2,3-dihydroxy-2-methylpropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-1-(2-hydroxypropyl)-5-methyl-3-oxo-2-phenyl-2,3-dihydro-1H-pyrazole-4-carboxamide;
N-(4-((6,7-bis(methyloxy)-4-quinazolinyl)oxy)-3-fluorophenyl)-1-(2-hydroxy-2-methylpropyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-1-(2-methyl-2-propen-1-yl)-3-oxo-2-phenyl-2,3-dihydro-1H-pyrazole-4-carboxamide;
N-(4-((6,7-bis(methyloxy)-4-quinolinyl)oxy)-3-fluorophenyl)-1-((2S)-2-hydroxypropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-5-methyl-3-oxo-1-(2-oxopropyl)-2-phenyl-2,3-dihydro-1H-pyrazole-4-carboxamide;
N-(4-((6,7-bis(methyloxy)-4-quinolinyl)oxy)-3-fluorophenyl)-1-(2,3-dihydroxy-2-methylpropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-5-methyl-1-(2-methyl-2-propen-1-yl)-3-oxo-2-phenyl-2,3-dihydro-1H-pyrazole-4-carboxamide;
N-(5-((6,7-bis(methyloxy)-4-quinolinyl)oxy)-2-pyridinyl)-5-methyl-3-oxo-2-phenyl-1-propyl-2,3-dihydro-1H-pyrazole-4-carboxamide;
N-(4-((6,7-bis(methyloxy)-4-quinolinyl)oxy)-3-fluorophenyl)-5-methyl-3-oxo-2-phenyl-2,3-dihydro-1H-pyrazole-4-carboxamide;
N-(5-((6,7-bis(methyloxy)-4-quinolinyl)oxy)-2-pyridinyl)-5-methyl-3-oxo-2-phenyl-1-(2-propen-1-yl)-2,3-dihydro-1H-pyrazole-4-carboxamide;
N-(4-((6,7-bis(methyloxy)-1-oxido-4-quinolinyl)oxy)-3-fluorophenyl)-5-methyl-3-oxo-2-phenyl-1-(2-propen-1-yl)-2,3-dihydro-1H-pyrazole-4-carboxamide;
N-(4-((6,7-bis(methyloxy)-4-quinolinyl)oxy)-3-fluorophenyl)-5-methyl-3-oxo-2-phenyl-1-(phenylmethyl)-2,3-dihydro-1H-pyrazole-4-carboxamide;
4-(6,7-dimethoxyquinolin-4-yloxy)-3-fluoro-N-(5-oxo-1-phenyl-2,5-dihydro-1H-pyrazol-3-yl)benzamide;
4-(6,7-dimethoxyquinolin-4-yloxy)-N-((1,2-dimethyl-5-oxo-3-phenyl-2,5-dihydro-1H-pyrazol-4-yl)methyl)-3-fluorobenzamide;
4-(6,7-dimethoxyquinolin-4-yloxy)-N-(2,3-dimethyl-5-oxo-1-phenyl-2,5-dihydro-1H-pyrazol-4-yl)-3-fluorobenzamide
4-(6,7-dimethoxyquinolin-4-yloxy)-N-(2,3-dimethyl-5-oxo-1-phenyl-2,5-dihydro-1H-pyrazol-4-yl)methyl)-3-fluorobenzamide;
1-benzyl-N-(5-(7-methoxyquinolin-4-yloxy)pyridin-2-yl)-2-oxo-1,2-dihydropyrazolo[1,5-a]pyridine-3-carboxamide;
4-((5-(6,7-dimethoxyquinolin-4-yloxy)pyridin-2-ylamino)methyl)-1,5-dimethyl-2-phenyl-1,2-dihydropyrazol-3-one;
N-(3-fluoro-4-(2-(3-methyl-1,2,4-oxadiazol-5-yl)thieno[3,2-b]pyridin-7-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-((2-(1-methyl-1H-imidazol-5-yl)thieno[3,2-b]pyridin-7-yl)oxy)phenyl)-1-(2-hydroxy-2-methylpropyl)-5-methyl-3-oxo-2-phenyl-2,3-dihydro-1H-pyrazole-4-carboxamide;
N-(3-fluoro-4-((2-(1-methyl-1H-imidazol-5-yl)thieno[3,2-b]pyridin-7-yl)oxy)phenyl)-1-(2R)-2-hydroxypropyl)-5-methyl-3-oxo-2-phenyl-2,3-dihydro-1H-pyrazole-4-carboxamide;
N-(3-fluoro-4-(7H-pyrrolo[2,3-d]pyrimidin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(1H-pyrrolo[2,3-b]pyridin-4-yloxy)phenyl)-1-(2-hydroxy-2-methylpropyl)-5-methyl-3-oxo-2-phenyl-2,3-dihydro-1H-pyrazole-4-carboxamide;
methyl(6-((4-(((1-(2-hydroxy-2-methylpropyl)-5-methyl-3-oxo-2-phenyl-2,3-dihydro-1H-pyrazol-4-yl)carbonyl)amino)phenyl)oxy)-1H-benzimidazol-2-yl)carbamate;
N-(4-(2-(azetidine-1-carbonyl)thieno[3,2-b]pyridin-7-yloxy)-3-fluorophenyl)-5-methyl-3-oxo-2-phenyl-1-propyl-2,3-dihydro-1H-pyrazole-4-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N-methylthieno[3,2-b]pyridine-2-carboxamide;
N-(3-fluoro-4-(2-(1-methylpiperazine-4-carbonyl)thieno[3,2-b]pyridin-7-yloxy)phenyl)-1-(2-hydroxy-2-methylpropyl)-5-methyl-3-oxo-2-phenyl-2,3-dihydro-1H-pyrazole-4-carboxamide;
N-(2-(dimethylamino)ethyl)-7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide;
N-(4-(2-(3-(dimethylamino)pyrrolidine-1-carbonyl)thieno[3,2-b]pyridin-7-yloxy)-3-fluorophenyl)-1-(2-hydroxy-2-methylpropyl)-5-methyl-3-oxo-2-phenyl-2,3-dihydro-1H-pyrazole-4-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N,N-dimethylthieno[3,2-b]pyridine-2-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide;
N-(2-(dimethylamino)ethyl)-7-(2-fluoro-4-(1-(2-hydroxy-2-methylpropyl)-5-methyl-3-oxo-2-phenyl-2,3-dihydro-1H-pyrazole-4-carboxamido)phenoxy)-N-methylthieno[3,2-b]pyridine-2-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N-(2-methoxyethyl)thieno[3,2-b]pyridine-2-carboxamide;
N-(4-(2-(azetidine-1-carbonyl)thieno[3,2-b]pyridin-7-yloxy)-3-fluorophenyl)-1-(2-hydroxy-2-methylpropyl)-5-methyl-3-oxo-2-phenyl-2,3-dihydro-1H-pyrazole-4-carboxamide;
N-cyclopropyl-7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide
7-(2-fluoro-4-(5-methyl-3-oxo-2-phenyl-1-propyl-2,3-dihydro-1H-pyrazole-4-carboxamido)phenoxy)thieno[3,2-b]pyridine-2-carboxamide;
N-(3-fluoro-4-(6-(pyrrolidine-1-carboxamido)pyrimidin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(6-(pyrrolidine-1-carboxamido)pyrimidin-4-yloxy)phenyl)-5-methyl-3-oxo-2-phenyl-1-propyl-2,3-dihydro-1H-pyrazole-4-carboxamide;
N-(6-(4-(1,5-dimethyl-3-oxo-2-phenyl-2,3-dihydro-1H-pyrazole-4-carboxamido)-2-fluorophenoxy)pyrimidin-4-yl)morpholine-4-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)morpholine-4-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)piperidine-1-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)-4-methylpiperazine-1-carboxamide;
(R)-N-(4-(6-(3-(dimethylamino)pyrrolidine-1-carboxamido)pyrimidin-4-yloxy)-3-fluorophenyl)-5-methyl-3-oxo-2-phenyl-1-propyl-2,3-dihydro-1H-pyrazole-4-carboxamide;
(R)-N-(4-(6-aminopyrimidin-4-yloxy)-3-fluorophenyl)-1-(2-hydroxypropyl)-5-methyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-1,5-dimethyl-3-oxo-2-phenyl-2,3-dihydro-1H-pyrazole-4-carboxamide;
N-(4-(4-(1,5-dimethyl-3-oxo-2-phenyl-2,3-dihydro-1H-pyrazole-4-carboxamido)-2-fluorophenoxy)pyridin-2-yl)piperidine-1-carboxamide;
(R)-N-(4-(2-(3-(dimethylamino)pyrrolidine-1-carboxamido)pyridin-4-yloxy)-3-fluorophenyl)-1,5-dimethyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-5-methyl-3-oxo-2-phenyl-1-propyl-2,3-dihydro-1H-pyrazole-4-carboxamide;
N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide;
N-(4-(2-fluoro-4-(1-(2-hydroxy-2-methylpropyl)-5-methyl-3-oxo-2-phenyl-2,3-dihydro-1H-pyrazole-4-carboxamido)phenoxy)pyridin-2-yl)piperidine-1-carboxamide;
5-methyl-N-(4-((7-(methyloxy)-4-quinolinyl)methyl)phenyl)-3-oxo-2-phenyl-1-propyl-2,3-dihydro-1H-pyrazole-4-carboxamide;
N-(4-(hydroxy(7-methoxyquinolin-4-yl)methyl)phenyl)-5-methyl-3-oxo-2-phenyl-1-propyl-2,3-dihydro-1H-pyrazole-4-carboxamide;
1,5-dimethyl-N-(5-((7-(methyloxy)-4-quinolinyl)oxy)-2-pyrimidinyl)-3-oxo-2-phenyl-2,3-dihydro-1H-pyrazole-4-carboxamide;
5-methyl-N-(4-((7-(methyloxy)-4-quinolinyl)sulfinyl)phenyl)-3-oxo-2-phenyl-1-propyl-2,3-dihydro-1H-pyrazole-4-carboxamide 1-(2-hydroxy-2-methylpropyl)-5-methyl-N-(4-((7-(methyloxy)-4-quinolinyl)thio)phenyl)-3-oxo-2-phenyl-2,3-dihydro-1H-pyrazole-4-carboxamide
5-methyl-N-(4-((7-(methyloxy)-4-quinolinyl)thio)phenyl)-3-oxo-2-phenyl-1-propyl-2,3-dihydro-1H-pyrazole-4-carboxamide 5-methyl-N-(3-((7-(methyloxy)-4-quinolinyl)oxy)propyl)-3-oxo-2-phenyl-1-propyl-2,3-dihydro-1H-pyrazole-4-carboxamide;
5-methyl-N-(trans-4-((7-(methyloxy)-4-quinolinyl)oxy)cyclohexyl)-3-oxo-2-phenyl-1-propyl-2,3-dihydro-1H-pyrazole-4-carboxamide;
5-methyl-N-(cis-4-((7-(methyloxy)-4-quinolinyl)oxy)cyclohexyl)-3-oxo-2-phenyl-1-propyl-2,3-dihydro-1H-pyrazole-4-carboxamide;
1-(2-hydroxy-2-methylpropyl)-5-methyl-N-(trans-4-((7-(methyloxy)-4-quinolinyl)oxy)cyclohexyl)-3-oxo-2-phenyl-2,3-dihydro-1H-pyrazole-4-carboxamide;
5-methyl-N-(4-((7-(methyloxy)-4-quinolinyl)amino)phenyl)-3-oxo-2-phenyl-1-propyl-2,3-dihydro-1H-pyrazole-4-carboxamide;
5-methyl-N-(5-((7-(methyloxy)-4-quinolinyl)oxy)-2-pyrimidinyl)-3-oxo-2-phenyl-1-propyl-2,3-dihydro-1H-pyrazole-4-carboxamide;
N-(3-fluoro-4-((7-(methyloxy)-4-quinolinyl)amino)phenyl)-1-(2-hydroxy-2-methylpropyl)-5-methyl-3-oxo-2-phenyl-2,3-dihydro-1H-pyrazole-4-carboxamide;
1-(2-hydroxy-2-methylpropyl)-5-methyl-4-((7-((7-(methyloxy)-4-quinolinyl)oxy)-2,3-dihydro-4H-1,4-benzoxazin-4-yl)carbonyl)-2-phenyl-1,2-dihydro-3H-pyrazol-3-one;
1-(2-hydroxy-2-methylpropyl)-5-methyl-N-(4-((7-(methyloxy)-4-quinolinyl)amino)phenyl)-3-oxo-2-phenyl-2,3-dihydro-1H-pyrazole-4-carboxamide;
N-(4-(6,7-dimethoxyquinolin-4-yloxy)-3-fluorophenyl)-3-hydroxy-2-(1-oxoisoindolin-2-yl)propanamide;
N-(4-(6,7-dimethoxyquinolin-4-yloxy)-3-fluorophenyl)-2-(1-oxoisoindolin-2-yl)acetamide;
N-(4-(6,7-dimethoxyquinolin-4-yloxy)-3-fluorophenyl)-2-oxo-1,5-diphenyl-1,2-dihydropyridine-3-carboxamide;
N-(5-((6,7-bis(methyloxy)-4-quinolinyl)oxy)-2-pyridinyl)-6-oxo-1-(phenylmethyl)-1,1′,2′,3′,6,6′-hexahydro-3,4′-bipyridine-5-carboxamide;
N-(5-((6,7-bis(methyloxy)-4-quinolinyl)oxy)-2-pyridinyl)-6-oxo-1-(phenylmethyl)-1,6-dihydro-3,3′-bipyridine-5-carboxamide;
N-(5-((6,7-bis(methyloxy)-4-quinolinyl)oxy)-2-pyridinyl)-6′-oxo-1′-(phenylmethyl)-1′,6′-dihydro-2,3′-bipyridine-5′-carboxamide
N-(5-((6,7-bis(methyloxy)-4-quinolinyl)oxy)-2-pyridinyl)-2-oxo-1-(phenylmethyl)-5-(2-thienyl)-1,2-dihydro-3-pyridinecarboxamide;
N-(5-((6,7-bis(methyloxy)-4-quinolinyl)oxy)-2-pyridinyl)-2-oxo-1-(phenylmethyl)-5-(2-pyrazinyl)-1,2-dihydro-3-pyridinecarboxamide;
N-(5-((6,7-bis(methyloxy)-4-quinolinyl)oxy)-2-pyridinyl)-5-methyl-2-oxo-1-(phenylmethyl)-1,2-dihydro-3-pyridinecarboxamide;
N-(4-((6,7-bis(methyloxy)-4-quinolinyl)oxy)-3-fluorophenyl)-5-bromo-1-(3-methylphenyl)-2-oxo-1,2-dihydro-3-pyridinecarboxamide;
N-(4-((6,7-bis(methyloxy)-4-quinolinyl)oxy)-3-fluorophenyl)-5-(1-methyl-1H-pyrazol-4-yl)-2-oxo-1-phenyl-1,2-dihydro-3-pyridinecarboxamide;
N-(3-fluoro-4-((6-(methyloxy)-743-(4-morpholinyl)propyl)oxy)-4-quinolinyl)oxy)phenyl)-2-oxo-5-phenyl-1-(phenylmethyl)-1,2-dihydro-3-pyridinecarboxamide;
1,1-dimethylethyl 5-(((5-((6,7-bis(methyloxy)-4-quinolinyl)oxy)-2-pyridinyl)amino)carbonyl)-6-oxo-1-(phenylmethyl)-1,3′,6,6′-tetrahydro-3,4′-bipyridine-1′(2′H)-carboxylate;
N-(4-((6,7-bis(methyloxy)-4-quinolinyl)oxy)-3-fluorophenyl)-2-oxo-1-(phenylmethyl)-5-(2-pyrimidinyl)-1,2-dihydro-3-pyridinecarboxamide;
N-(4-((6,7-bis(methyloxy)-4-quinolinyl)oxy)-3-fluorophenyl)-2-oxo-1-phenyl-5-(1H-pyrazol-4-yl)-1,2-dihydro-3-pyridinecarboxamide;
1-benzyl-5-bromo-N-(2-chloro-4-(6,7-dimethoxyquinolin-4-yloxy)phenyl)-2-oxo-1,2-dihydropyridine-3-carboxamide;
N-(5-(7-methoxyquinolin-4-yloxy)pyridin-2-yl)-2-oxo-1-phenyl-5-(pyridin-3-yl)-1,2-dihydropyridine-3-carboxamide;
N-(5-(7-methoxyquinolin-4-yloxy)pyridin-2-yl)-2-oxo-1-phenyl-5-(pyrazin-2-yl)-1,2-dihydropyridine-3-carboxamide;
N-(5-(6,7-dimethoxyquinolin-4-yloxy)pyridin-2-yl)-2-oxo-1-phenyl-5-(pyridin-3-yl)-1,2-dihydropyridine-3-carboxamide;
N-(5-(6,7-dimethoxyquinolin-4-yloxy)pyridin-2-yl)-2-oxo-1-phenyl-5-(pyrazin-2-yl)-1,2-dihydropyridine-3-carboxamide;
N-(5-(6,7-dimethoxyquinolin-4-yloxy)pyridin-2-yl)-2-oxo-1-phenyl-5-(thiophen-2-yl)-1,2-dihydropyridine-3-carboxamide;
5-benzyl-N-(5-(6,7-dimethoxyquinolin-4-yloxy)pyridin-2-yl)-2-oxo-1-phenyl-1,2-dihydropyridine-3-carboxamide;
tert-butyl 4-(5-((5-(6,7-dimethoxyquinolin-4-yloxy)pyridin-2-yl)carbamoyl)-6-oxo-1-phenyl-1,6-dihydropyridin-3-yl)-5,6-dihydropyridine-1(2H)-carboxylate;
5-bromo-N-(2-chloro-4-(6,7-dimethoxyquinolin-4-yloxy)phenyl)-2-oxo-1-phenyl-1,2-dihydropyridine-3-carboxamide;
N-(5-(6,7-dimethoxyquinolin-4-yloxy)pyridin-2-yl)-4-(2-methoxyethylamino)-2-oxo-1-phenyl-1,2-dihydropyridine-3-carboxamide;
N-(5-(6,7-dimethoxyquinolin-4-yloxy)pyridin-2-yl)-2-oxo-1-phenyl-4-(tetrahydro-2H-pyran-4-ylamino)-1,2-dihydropyridine-3-carboxamide;
N-(5-(6,7-dimethoxyquinolin-4-yloxy)pyridin-2-yl)-2-oxo-1-phenyl-4-(phenylamino)-1,2-dihydropyridine-3-carboxamide;
N-(5-(6,7-dimethoxyquinolin-4-yloxy)pyridin-2-yl)-4-(4-methylpiperazin-1-yl)-2-oxo-1-phenyl-1,2-dihydropyridine-3-carboxamide;
N-(5-(6,7-dimethoxyquinolin-4-yloxy)pyridin-2-yl)-4-(methylamino)-2-oxo-1-phenyl-1,2-dihydropyridine-3-carboxamide;
N-(5-(6,7-dimethoxyquinolin-4-yloxy)pyridin-2-yl)-4-(dimethylamino)-2-oxo-1-phenyl-1,2-dihydropyridine-3-carboxamide;
4-(2-methoxyethylamino)-N-(5-(7-methoxyquinolin-4-yloxy)pyridin-2-yl)-2-oxo-1-phenyl-1,2-dihydropyridine-3-carboxamide;
N-(3-fluoro-4-(7-methoxyquinolin-4-yloxy)phenyl)-4-(2-methoxyethylamino)-2-oxo-1-phenyl-1,2-dihydropyridine-3-carboxamide;
N-(4-((6,7-bis(methyloxy)-4-quinolinyl)oxy)-3-fluorophenyl)-1-cyclopentyl-6-oxo-5-(2-oxo-1-pyrrolidinyl)-1,6-dihydro-3-pyridinecarboxamide;
1-benzyl-N-(5-(6,7-dimethoxyquinolin-4-yloxy)pyridin-2-yl)-4-(2-methoxyethylamino)-2-oxo-1,2-dihydropyridine-3-carboxamide;
1-benzyl-N-(5-(6,7-dimethoxyquinolin-4-yloxy)pyridin-2-yl)-4-(dimethylamino)-2-oxo-1,2-dihydropyridine-3-carboxamide;
1-benzyl-N-(5-(6,7-dimethoxyquinolin-4-yloxy)pyridin-2-yl)-4-(methylamino)-2-oxo-1,2-dihydropyridine-3-carboxamide;
1-benzyl-N-(5-(6,7-dimethoxyquinolin-4-yloxy)pyridin-2-yl)-2-oxo-4-(phenylamino)-1,2-dihydropyridine-3-carboxamide;
1-benzyl-N-(5-(6,7-dimethoxyquinolin-4-yloxy)pyridin-2-yl)-2-oxo-4-(pyridin-4-ylamino)-1,2-dihydropyridine-3-carboxamide;
1-benzyl-N-(5-(6,7-dimethoxyquinolin-4-yloxy)pyridin-2-yl)-4-(4-methylpiperazin-1-yl)-2-oxo-1,2-dihydropyridine-3-carboxamide;
1-benzyl-N-(5-(6,7-dimethoxyquinolin-4-yloxy)pyridin-2-yl)-2-oxo-4-(tetrahydro-2H-pyran-4-ylamino)-1,2-dihydropyridine-3-carboxamide;
1-benzyl-N-(5-(6,7-dimethoxyquinolin-4-yloxy)pyridin-2-yl)-2-oxo-4-(4-(trifluoromethyl)phenylamino)-1,2-dihydropyridine-3-carboxamide;
1-cyclopentyl-N-(4-(6,7-dimethoxyquinolin-4-yloxy)-3-fluorophenyl)-6-oxo-5-(2-oxopyrrolidin-1-yl)-1,6-dihydropyridine-3-carboxamide;
N-(3-fluoro-4-(2-(pyrrolidine-1-carboxamido)pyridin-4-yloxy)phenyl)-3-oxo-2-phenyl-2,3-dihydropyridazine-4-carboxamide;
6-((diethylamino)methyl)-N-(4-(6,7-dimethoxyquinolin-4-yloxy)-3-fluorophenyl)-3-oxo-2-phenyl-2,3-dihydropyridazine-4-carboxamide;
6-((dimethylamino)methyl)-N-(3-fluoro-4-(7-methoxyquinolin-4-yloxy)phenyl)-3-oxo-2-phenyl-2,3-dihydropyridazine-4-carboxamide;
N-(3-fluoro-4-(7-methoxyquinolin-4-yloxy)phenyl)-6-methyl-3-oxo-2-phenyl-2,3-dihydropyridazine-4-carboxamide;
N-(5-(6,7-dimethoxyquinolin-4-yloxy)pyridin-2-yl)-6-methyl-3-oxo-2-phenyl-2,3-dihydropyridazine-4-carboxamide;
2-benzyl-N-(5-(6,7-dimethoxyquinolin-4-yloxy)pyridin-2-yl)-6-methyl-3-oxo-2,3-dihydropyridazine-4-carboxamide;
N-(3-fluoro-4-(7-methoxyquinolin-4-yloxy)phenyl)-3-oxo-2-phenyl-2,3-dihydropyridazine-4-carboxamide;
N-(2-chloro-4-(6,7-dimethoxyquinolin-4-yloxy)phenyl)-6-methyl-3-oxo-2-phenyl-2,3-dihydropyridazine-4-carboxamide;
(R)-N-(4-(6,7-dimethoxyquinolin-4-yloxy)-3-fluorophenyl)-6-((3-(dimethylamino)pyrrolidin-1-yl)methyl)-3-oxo-2-phenyl-2,3-dihydropyridazine-4-carboxamide;
3-benzyl-N-(4-(6,7-dimethoxyquinolin-4-yloxy)-3-fluorophenyl)-2-oxoimidazolidine-1-carboxamide;
N-(4-(6,7-dimethoxyquinolin-4-yloxy)-3-fluorophenyl)-5-((dimethylamino)methyl)-2-oxo-3-phenyl-tetrahydropyrimidine-1(2H)-carboxamide;
N-(3-fluoro-4-(7-methoxyquinolin-4-yloxy)phenyl)-3-oxo-4-phenylmorpholine-2-carboxamide;
N-(5-(7-methoxyquinolin-4-yloxy)pyridin-2-yl)-1-methyl-3-oxo-2-phenyl-2,3-dihydro-1H-pyrazole-4-carboxamide; and
N-(3-fluoro-4-(7-methoxyquinolin-4-yloxy)phenyl)-3-oxo-4-phenylmorpholine-2-carboxamide.

15. The method of claim 1, wherein the HGF/SF:c-Met inhibitor is selected from the group consisting of Amgen Compound 2, Amgen Compound 3, ARQ197, MK2461, MK 8033, PF04217903, PF2341066, JNJ38877605, XL880, XL184, MGCD265, BMS 777607 and E7050.

16. The method of claim 1, wherein the cancer is selected from lung cancer, breast cancer, colon cancer, kidney cancer and head and neck cancer.

17. The method of claim 16, wherein the lung cancer is non-small cell lung cancer.

18. The method of claim 16, wherein the kidney cancer is renal cell carcinoma.

19. The method of claim 16, wherein the head and neck cancer is Glioblastoma Multiforme.

20. A kit comprising, in one or more containers, separately or in admixture one or more HGF/SF:c-Met inhibitors and one or more VEGF inhibitors.
